(19) United States
(12) Patent Application Publication
(10) Pub. No.: US 2024/0307548 A1
(43) Pub. Date: Sep. 19, 2024

(54) ENHANCED TARGETING USING ANTIBODY-OLIGONUCLEOTIDE CONJUGATES

(71) Applicants: Code Biotherapeutics, Inc., Hatfield, PA (US); University of Maryland, College Park, College Park, MD (US)

(72) Inventors: Silvia Muro, Barcelona, PA (US); Niksa Roki, Lewis Center, OH (US); Robert Getts, Hatfield, PA (US)

(21) Appl. No.: 18/279,938

(22) PCT Filed: Mar. 4, 2022
(86) PCT No.: PCT/US2022/018855

§ 371 Date: Sep. 1, 2023

Related U.S. Application Data
(60) Provisional application No. 63/156,869, filed on Mar. 4, 2021.

(51) Int. Cl. A61K 47/68; A61K 9/00; A61K 39/00; A61K 47/42; C07K 16/28; C07K 19/00

(52) U.S. Cl. CPC A61K 47/6807; A61K 9/0019; A61K 47/42; A61K 47/6849; C07K 16/2821; C07K 19/00; A61K 2039/505; C07K 2317/40; C07K 2317/92; C07K 2317/94; C12N 2310/3513

(57) ABSTRACT

The present disclosure provides antibodies, and fragments thereof, conjugated to an oligonucleotide, wherein the oligonucleotide is not hybridized to a DNA dendrimer, compositions comprising the same, and uses thereof in methods of detection, methods of cell isolation, methods of depletion, methods of diagnosis, and methods of treatment.

### CROSS REFERENCE TO RELATED APPLICATIONS

This application is a 35 U.S.C. § 371 National Stage application of International Application Number PCT/US2022/018855, filed Mar. 4, 2022, which claims priority to U.S. Provisional Application No. 63/156,869, filed Mar. 4, 2021, each of which is hereby incorporated by reference in their entirety.

### REFERENCE TO SEQUENCE LISTING

This application includes a Sequence Listing filed electronically as a text file named 19128800602SEQ, created on Mar. 2, 2022, with a size of 1 kilobyte. The Sequence Listing is incorporated herein by reference.

### FIELD

The present disclosure is directed to antibodies, and fragments thereof, conjugated to an oligonucleotide, wherein the oligonucleotide is not hybridized to a DNA dendrimer, compositions comprising the same, and uses thereof in methods of detection, methods of cell isolation, methods of depletion, methods of diagnosis, and methods of treatment.

### BACKGROUND

Antibodies have been widely used as therapeutic compounds for the treatment of numerous diseases and conditions such as, for example, cancer, autoimmunity, and inflammatory diseases. In addition, antibodies have also been used for drug delivery to target antigens. Therapeutic antibodies recognize and bind to antigen receptors to activate or inhibit a series of biological processes for blocking cancer cell growth or triggering the immune system. Therapeutic antibodies possess characteristic properties of high affinity, high specificity, and low immunogenicity. Early monoclonal antibodies (mab) were generated by fusing myeloma cells and spleen cells to obtain hybridomas secreting mab. Early therapeutic antibodies were usually produced in murine hosts, leading to serious human anti-mouse antibody expression, which can interfere with desired therapeutic effects. The disadvantages of short half-life, xenogenicity, and limited activity have been gradually improved with the development of gene engineering, in vitro cell culture technology, and a better understanding of antibody function. In the past four decades, therapeutic antibodies have improved via antibody engineering. Additional improvements in antibody function and/or activity is still desired.

### SUMMARY

The present disclosure provides an antibody, or antigen-binding fragment thereof, conjugated to an oligonucleotide, wherein the oligonucleotide is not hybridized to a DNA dendrimer, wherein: the antibody, or antigen-binding fragment thereof, binds an antigen to a greater extent or faster than an identical antibody, or antigen-binding fragment thereof, that is not conjugated to an identical oligonucleotide, and wherein the identical oligonucleotide is not hybridized to an identical DNA dendrimer; or upon intravenous administration of a physiologically acceptable amount of the antibody, or antigen-binding fragment thereof, to a subject, the antibody, or antigen-binding fragment thereof, is present in the blood of the subject to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

The present disclosure also provides compositions comprising the antibody, or antigen-binding fragment thereof.

The present disclosure also provides methods of increasing an extent to which an antibody, or antigen binding fragment thereof, binds an antigen, the method comprising conjugating an antibody, or antigen-binding fragment thereof, which is in need thereof, to an oligonucleotide, wherein the oligonucleotide is not hybridized to a DNA dendrimer, wherein the antibody, or antigen-binding fragment thereof, binds an antigen to a greater extent or faster than an identical antibody, or antigen-binding fragment thereof, not conjugated to an identical oligonucleotide, and wherein the identical oligonucleotide is not hybridized to an identical DNA dendrimer.

The present disclosure also provides methods of decreasing non-specific binding of an antibody, or antigen-binding fragment thereof, the method comprising conjugating an antibody, or antigen-binding fragment thereof, which is in need thereof, to an oligonucleotide, wherein the oligonucleotide is not hybridized to a DNA dendrimer, wherein, upon intravenous administration of a physiologically acceptable amount of the antibody, or antigen-binding fragment thereof, to a subject, the antibody, or antigen-binding fragment thereof, is present in the subject's blood to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

The present disclosure also provides methods of treating or preventing a disease in a subject, the method comprising administering to a subject in need thereof a physiologically acceptable amount of an antibody, or antigen-binding fragment thereof, conjugated to an oligonucleotide, wherein the oligonucleotide is not hybridized to a DNA dendrimer, wherein: the antibody, or antigen-binding fragment thereof, binds an antigen to a greater extent or faster than an identical antibody, or antigen-binding fragment thereof, that is not conjugated to an identical oligonucleotide, and wherein the identical oligonucleotide is not hybridized to an identical DNA dendrimer; or upon intravenous administration of a physiologically acceptable amount of the antibody, or antigen-binding fragment thereof, to a subject, the antibody, or antigen-binding fragment thereof, is present in the subject's blood to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

### BRIEF DESCRIPTION OF THE DRAWINGS

It is to be understood that both the foregoing general description and the following detailed description are exemplary and explanatory only and are not restrictive of the embodiments, as claimed herein.

The patent or application file contains at least one drawing executed in color. Copies of this patent or patent application publication with color drawing(s) will be provided by the Office upon request and payment of the necessary fee.

FIG. 1 shows IgG-oligo enhances removal from the bloodstream. Faster clearance from the circulation for non-specific IgG-oligo compared to IgG is observed, which may help reduce possible systemic side effects associated with the use of antibodies.

FIG. 2 shows IgG-oligo enhances spleen and liver clearance. Increased liver and spleen accumulation of non-specific IgG-oligo compared to IgG is observed, which can be due to the faster removal from the circulation, shown in FIG. 1, into these organs that are in charge of clearance, which in turn can help attenuate possible side effects derived from using antibodies on non-clearance organs. Also, there is a decreased level of IgG-oligo in the bladder, kidney, and stomach. This may be due to removal taking place in the spleen and liver, where levels are higher, or because excretion through the renal and gastric routes may be faster and IgG-oligo is no longer present in these organs, which is also adequate to remove the antibody from the body and avoid side effects.

FIG. 3 shows anti-ICAM-oligo is rapidly removed from the bloodstream. Specific anti-ICAM antibody shows faster clearance from circulation when conjugated to oligo compared to non-oligo anti-ICAM, which should also serve to protect from systemic side effects, as described above. In addition, faster clearance of anti-ICAM-oligo compared to IgG-oligo indicates that this protective effect is more apparent for an antibody that recognizes a specific antigen. This is relevant as specific antibodies are the ones recognizing antigens, therefore, the ones intended to be used in a research, diagnostic, preventive, or therapeutic setting.

FIG. 4 shows anti-ICAM-oligo enhances lung targeting. Specific anti-ICAM antibody targets the lungs (main site for ICAM-1 expression) markedly better compared to anti-ICAM control and much greater than non-specific IgG (conjugated or not to oligo), demonstrating enhanced absolute targeting and specificity.

FIG. 5 shows lung targeting of anti-ICAM-oligo is highly specific compared to other formulations and provided minimal accumulation in lymph nodes. Enhanced specificity toward the main site for ICAM-1 expression (the lungs) offered by anti-ICAM-oligo is shown as compared to all other species injected.

### DESCRIPTION OF EMBODIMENTS

DNA oligonucleotides may be coupled to antibodies for several purposes and applications which may involve analytical in vitro systems, ex vivo tissues, cell cultures, or in vivo organisms, such as: a) an oligonucleotide can be used as a linker between an antibody and a diagnostic or therapeutic cargo (antibody-probe or antibody-drug conjugates); b) an oligonucleotide can be used as a linker between an antibody and a drug delivery vehicle (e.g., liposome, polymer, dendrimer, inorganic nanoparticle, etc.) which is loaded with a diagnostic or therapeutic cargo; c) an oligonucleotide can be (fully or partially) annealed to a complementary oligonucleotide and the resulting double stranded DNA moiety can be loaded with DNA intercalating drugs (e.g., doxorubicin); d) an oligonucleotide can be annealed with complementary oligonucleotide where the resulting double stranded DNA or DNA/RNA chimera can express or block expression of one/a group of gene(s) or messenger RNA(s) (e.g., siRNA, microRNA effects, etc.); e) an oligonucleotide can be designed which such a sequence as to provide on its own the functions described in d); f) an oligonucleotide can be designed with such a sequence as to fold into an aptamer and provide specific binding to the a different antigen than the antibody to which the oligonucleotide is attached, or same antigen as the antibody to increase its affinity or to activate/block the antigen at the same/different domains. Hence, these designs could be used as tools in research or could be applied to the diagnostics and/or therapeutics fields.

Coupling of a monoclonal antibody which recognizes ICAM⁻¹, a cell surface protein mainly overexpressed in the Jung but also other organs in diseases that involve inflammation, to a DNA oligonucleotide was observed to markedly increase binding of the antibody to ICAM-1 target sites (mainly the lungs) in the body after intravenous injection in mice. In addition, antibody-oligonucleotide conjugates were cleared from the circulation in mice faster than regular antibodies and were distributed more profusely, which may serve to avoid systemic side effects of antibodies applied to the human body. This observation was surprising since coupling to an oligonucleotide would be expected to lower the binding and/or targeting ability of the antibody because of: a) steric hindrance, b) possible modification or interference with the variable region which recognizes the antigen, c) changes in the physicochemical properties of other antibody regions, and/or d) the negative charge provided by the oligonucleotide (nucleic acids are negatively charged species) that would repel the negative charge present on the surface of cells in the body (provided by sugar moieties of glycosylated proteins and lipids in the cellular membrane). The mechanism for this unexpected finding is unknown; nevertheless, the finding holds significant translational potential.

The terminology used herein is for the purpose of describing particular embodiments only and is not intended to be limiting. Various terms relating to aspects of the disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in any specific order. Where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by +10% and remain within the scope of the disclosed embodiments.

As used herein, the term “antibody” refers to an immunoglobulin molecule that specifically binds to, or is immunologically reactive with, a particular antigen, and includes polyclonal, monoclonal, genetically engineered and otherwise modified forms thereof including, but not limited to, nanobodies, chimeric antibodies, humanized antibodies, heteroconjugate antibodies (e.g., bispecific antibodies, diabodies, triabodies, and tetrabodies).

As used herein, the phrase “antigen-binding fragment thereof” means a fragment of an antibody that is able to bind an antigen. Antigen-binding fragments include, but are not limited to, Fab, Fab′, F(ab′)₂, Fv, scFv, scFv-Fc, diabody, bispecific diabody, trispecific triabody, minibody, monospecific Fab₂, bispecific Fab₂, trispecific Fab₃, nanobody, IgNAR, V-NAR, hcIgG, and VhH fragments.

As used herein, the phrase “chimeric antibody” refers to an antibody having variable sequences derived from a non-human immunoglobulin, such as rat or mouse antibody, and human immunoglobulin constant regions, typically chosen from a human immunoglobulin template.

As used herein, the term “Fv” fragment is the minimum antibody fragment that contains a complete target recognition and binding site. This region consists of a dimer of one heavy and one light chain variable domain in a noncovalent association (V_{H}-V_{L} dimer). It is in this configuration that the three CDRs of each variable domain interact to define a target binding site on the surface of the V_{H}-V_{L} dimer.

As used herein, the phrase “human antibodies” refers to antibodies having the amino acid sequence of a human immunoglobulin and includes antibodies isolated from human immunoglobulin libraries or from animals transgenic for one or more human immunoglobulins and that do not express endogenous immunoglobulins.

As used herein, the phrase “humanized antibody” refers to a chimeric antibody, or an antigen-binding fragment thereof, which contain minimal sequences derived from non-human immunoglobulin. In general, the humanized antibody will comprise substantially all of at least one, and typically two, variable domains, in which all or substantially all of the CDR regions correspond to those of a non-human immunoglobulin and all or substantially all of the framework (FR) regions that are those of a human immunoglobulin sequence. The humanized antibody can also comprise at least a portion of an immunoglobulin constant region (Fc), typically that of a human immunoglobulin consensus sequence.

As used herein, the phrase “in need thereof” means that the “subject” or “patient” has been identified as having a need for the particular method, prevention, or treatment. In some embodiments, the identification can be by any means of diagnosis. In any of the methods, preventions, and treatments described herein, the “subject” or “patient” can be in need thereof.

As used herein, a “nucleic acid molecule” is a polymeric form of nucleotides of any length, may comprise DNA and/or RNA, and can be single-stranded, double-stranded, or multiple stranded. One strand of a nucleic acid also refers to its complement.

As used herein, the phrase “primatized antibody” refers to an antibody comprising monkey variable regions and human constant regions.

As used herein, the phrase “regulatory sequence” is intended to include promoters, enhancers and other expression control elements, such as polyadenylation signals, that control the transcription or translation of the antibody chain genes.

As used herein, the term “single chain Fv” or “scFv” refers to a single chain Fv antibody in which the variable domains of the heavy chain and the light chain (V_{H} and V_{L} domains) from a traditional antibody have been joined to form one chain. Generally, the Fv polypeptide further comprises a polypeptide linker between the V_{H} and V_{L} domains which enables the scFv to form the desired structure for target binding.

As used herein, the terms “subject” and “patient” are used interchangeably. A subject includes any animal, including mammals. Mammals include, but are not limited to, farm animals (e.g., horse, cow, pig), companion animals (e.g., dog, cat), laboratory animals (e.g., mouse, rat, rabbits), and non-human primates (e.g., monkey). In some embodiments, the subject or patient is a human.

As used herein, the term “V_{H}” refers to the variable region of an immunoglobulin heavy chain of an antibody, including the heavy chain of, for example, an Fv, scFv, or Fab fragment.

As used herein, the term “V_{L}” refers to the variable region of an immunoglobulin light chain of an antibody, including the light chain of, for example, an Fv, scFv, dsFv, or Fab fragment.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “internal portion” of an antibody, or antigen-binding fragment thereof, refers to an amino acid other than the amino acids at the N- and C-terminals of the antibody, or antigen-binding fragment thereof, respectively.

As used herein, the term “therapeutic antibody” refers to an antibody, or antigen-binding fragment thereof, suitable for administration to a subject to treat and/or prevent a disease or disorder.

As used herein. a “DNA dendrimer” is a highly ordered, branched polymeric molecule having at least three DNA strands. One of the strands is at least partially complementary to one of the other strands for at least ten contiguous nucleotides.

As used herein, a first oligonucleotide is “hybridized” to a second oligonucleotide when at least ten contiguous nucleotides in the first oligonucleotide are bound by Crick-Watson base pairing to at least ten contiguous, complimentary nucleotides in the second nucleotides.

As used herein, a first antibody, or antigen-binding fragment thereof, binds an antigen “to a greater extent or faster” than a second antibody, or antigen-binding fragment thereof, when the first antibody, or antigen-binding fragment thereof, has increased affinity, an increase in a binding constant, an increase in K_{A} (kₒₙ/k_{off}), or an increase in avidity for an antigen than the second antibody, or antigen-binding fragment thereof.

As used herein, a first antibody, or antigen-binding fragment thereof, is “identical” to a second antibody, or antigen-binding fragment thereof, when the first antibody, or antigen-binding fragment thereof, and the second antibody, or antigen-binding fragment thereof: (i) have the same amino acid sequence(s); (ii) the same species of derivatization(s), if any; (iii) have the same species of covalent linkage to the same species of effector moiety, if any; (iv) are fused via the same species of covalent bond to the same species of amino acid sequence of the same species of another protein or portion thereof, if any; (v) are conjugated to the same species of small molecule toxin, if any; (vi) are conjugated to the same species of liposome, if any; (vii) are linked to the same species of polyethylene glycol (PEG) moiety, if any; (viii) have the same species of modification(s), if any; and/or (ix) are coupled to the same species of detectable substance(s). However, a first antibody, or antigen-binding fragment thereof, and a second antibody, or antigen-binding fragment thereof, may be identical but for at least one specified difference. By way of a non-limiting example, a first antibody, or antigen-binding fragment thereof, and a second antibody, or antigen-binding fragment thereof, may be identical but for a specified difference in an amino acid sequence.

As used herein, a first oligonucleotide is “identical” to a second oligonucleotide are identical when the first oligonucleotide and the second oligonucleotides: (i) have the same nucleotide sequence(s); and/or (ii) the oligonucleotide comprised the same species of non-natural nucleotide(s), modified nucleotide(s), nucleotide analog(s), or nucleotide substitute(s) at the same position(s). However, a first oligonucleotide and a second oligonucleotide may be identical but for a specified difference. By way of a non-limiting example, a first oligonucleotide and a second oligonucleotide may be identical but for a specified difference in a nucleotide sequence.

For example, attaching an oligonucleotide to a therapeutic antibody, such as a checkpoint inhibitor, EGFR, Her2, Herceptin, or an antibody-probe or antibody-drug conjugate may significantly improve the localization and binding to a target or solubility of the antibody, thus, improving the diagnostic or therapeutic index of the agent while reducing toxicity because a lower dose could be administered or because of more efficient clearing and avoidance of off-target effects. This same result may be observed where an antibody fragment or scFv is used with a diagnostic probe and/or a therapeutic agent. In certain embodiments, antibody-like molecules are also contemplated, including, but not limited to, minibodies, nanobodies, diabodies, triabodies, and tetrabodies. In certain embodiments, antibody-based molecules are also contemplated, including, but not limited to, bispecific-, trispecific-, or tetraspecific-antibodies.

The present disclosure provides an antibody, or antigen-binding fragment thereof, conjugated to an oligonucleotide, wherein the oligonucleotide is not hybridized to a DNA dendrimer, wherein: the antibody, or antigen-binding fragment thereof, binds an antigen to a greater extent or faster than an identical antibody, or antigen-binding fragment thereof, that is not conjugated to an identical oligonucleotide, and wherein the identical oligonucleotide is not hybridized to an identical DNA dendrimer; or upon intravenous administration of a physiologically acceptable amount of the antibody, or antigen-binding fragment thereof, to a subject, the antibody, or antigen-binding fragment thereof, is present in the blood of the subject to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

The present disclosure also provides methods of increasing an extent or speed to which an antibody, or antigen binding fragment thereof, binds an antigen, the method comprising conjugating an antibody, or antigen-binding fragment thereof, which is in need thereof, to an oligonucleotide, wherein the oligonucleotide is not hybridized to a DNA dendrimer, wherein the antibody, or antigen-binding fragment thereof, binds an antigen to a greater extent or faster than an identical antibody, or antigen-binding fragment thereof, not conjugated to an identical oligonucleotide, and wherein the identical oligonucleotide is not hybridized to an identical DNA dendrimer.

The present disclosure also provides methods of decreasing non-specific binding of an antibody, or antigen-binding fragment thereof, the method comprising conjugating an antibody, or antigen-binding fragment thereof, which is in need thereof, to an oligonucleotide, wherein the oligonucleotide is not hybridized to a DNA dendrimer, wherein, upon intravenous administration of a physiologically acceptable amount of the antibody, or antigen-binding fragment thereof, to a subject, the antibody, or antigen-binding fragment thereof, is present in the subject's blood to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

The present disclosure also provides methods of decreasing non-specific binding of an antibody, or antigen-binding fragment thereof, the method comprising conjugating an antibody, or antigen-binding fragment thereof, which is in need thereof, to an oligonucleotide, wherein the oligonucleotide is not hybridized to a DNA dendrimer, wherein, upon intravenous administration of a physiologically acceptable amount of the antibody, or antigen-binding fragment thereof, to a subject, the antibody, or antigen-binding fragment thereof, binds its target to a greater extent or faster than an identical antibody, or antigen-binding fragment thereof, not conjugated to an identical oligonucleotide, wherein the identical oligonucleotide is not hybridized to an identical DNA dendrimer.

The present disclosure also provides methods of diagnosing, treating, or preventing a disease in a subject, the method comprising administering to a subject in need thereof a physiologically acceptable amount of an antibody, or antigen-binding fragment thereof, conjugated to an oligonucleotide, wherein the oligonucleotide is not hybridized to a DNA dendrimer, wherein: the antibody, or antigen-binding fragment thereof, binds an antigen to a greater extent or faster than an identical antibody, or antigen-binding fragment thereof, that is not conjugated to an identical oligonucleotide, and wherein the identical oligonucleotide is not hybridized to an identical DNA dendrimer; or upon intravenous administration of a physiologically acceptable amount of the antibody, or antigen-binding fragment thereof, to a subject, the antibody, or antigen-binding fragment thereof, is present in the subject's blood to a lesser extent at a period of time after intravenous administration than an identical antibody, or antigen-binding fragment thereof, not conjugated to an identical oligonucleotide, wherein the identical oligonucleotide is not hybridized to an identical DNA dendrimer.

The antibodies, or antigen-binding fragments thereof, can be any isotype. In some embodiments, the antibody is an IgM, IgA, IgD, IgE, or IgG antibody. In some embodiments, the antibody is an IgM antibody. In some embodiments, the antibody is an IgG antibody. In some embodiments, the antibody is an IgG1, IgG2a, IgG2b, IgG3, or IgG4 antibody. In some embodiments, the IgG1 antibody is an IgG1 Glm1, Glm2, Glm3, Glm17, nGlm1, nGlm2, or nGlm17 allotype antibody. In some embodiments, the antibody is an IgG1 Glm17 allotype antibody. In some embodiments, the IgA antibody is an IgA1 or IgA2 antibody. In some embodiments, the antibody has a variant heavy chain.

In some embodiments, the variant IgG1 heavy chain is paired with a kappa light chain of allotype Km1, Km2, or Km3. In some embodiments, the variant IgG1 heavy chain is paired with a lambda light chain.

In some embodiments, the antibodies, or antigen-binding fragments thereof, are humanized and IgG. In some embodiments, the antibodies, or antigen-binding fragments thereof, are humanized and IgG1.

In some embodiments, the antibody, or antigen-binding fragment thereof, is a chimeric antibody, or antigen-binding fragment thereof. Methods for producing chimeric antibodies are known in the art (see, Morrison, Science, 1985, 229, 1202-1207; Oi et al., BioTechniques, 1986, 4, 214-221; Gillies et al., J. Immunol. Methods, 1985, 125, 191-202; and U.S. Pat. Nos. 5,807,715, 4,816,567, and 4,816,397).

In some embodiments, the chimeric antibody, or antigen-binding fragment thereof, is a primatized chimeric antibody, or antigen-binding fragment thereof. Methods for producing primatized antibodies are known in the art (see, U.S. Pat. Nos. 5,658,570, 5,681,722, and 5,693,780).

In some embodiments, the chimeric antibody, or antigen-binding fragment thereof, is a humanized antibody, or antigen-binding fragment thereof. Methods for producing humanized antibodies are known in the art (see, Riechmann et al., Nature, 1988, 332, 323-327; Padlan, Mol. Immunol., 1991, 28, 489-498; Studnicka et al., Prot. Eng., 1994, 7, 805-814; Roguska et al., Proc. Natl. Acad. Sci., 1994, 91, 969-973; European Patent Nos. EP239400, EP592106, and EP519596; PCT Publication WO 91/09967; and U.S. Pat. Nos. 5,225,539, 5,530,101, 5,585,089, 5,693,761, 5,693,762, 6,180,370, and 5,565,332).

In some embodiments, the antibody, or antigen-binding fragment thereof, is completely humanized. Completely “human” antibodies can be desirable for therapeutic treatment of human patients. Methods for producing completely human antibodies are known in the art, including phage display methods using antibody libraries derived from human immunoglobulin sequences (see, U.S. Pat. Nos. 4,444,887 and 4,716,111; and PCT Publications WO 98/46645, WO 98/50433, WO 98/24893, WO 98/16654, WO 96/34096, WO 96/33735, and WO 91/10741). Human antibodies can also be produced using transgenic mice which are incapable of expressing functional endogenous immunoglobulins, but which can express human immunoglobulin genes (see, PCT Publications WO 98/24893, WO 92/01047, WO 96/34096, and WO 96/33735; and U.S. Pat. Nos. 5,413,923, 5,625,126, 5,633,425, 5,569,825, 5,661,016, 5,545,806, 5,814,318, 5,885,793, 5,916,771, and 5,939,598). Completely human antibodies that recognize a selected epitope can also be generated using a technique referred to as “guided selection” (see, Jespers et al., Biotechnology, 1988, 12, 899-903).

In some embodiments, the antibodies, or antigen-binding fragments thereof, are bispecific antibodies. Bispecific antibodies have binding specificities for at least two different antigens (i.e., one of the binding specificities is directed to a one antigen, and the other binding specificity is for any other antigen, such as a cell-surface protein, receptor, receptor subunit, tissue-specific antigen, virally derived protein, virally encoded envelope protein, bacterially derived protein, or bacterial surface protein, etc.).

In some embodiments, the antibodies, or antigen-binding fragments thereof, are derivatized antibodies. For example, the derivatized antibodies can be antibodies modified by glycosylation, acetylation, pegylation, phosphorylation, amidation, derivatization by known protecting/blocking groups, proteolytic cleavage, linkage to a cellular ligand or other protein, and the like. Any of numerous chemical modifications can be carried out by known techniques, including, but not limited to, specific chemical cleavage, acetylation, formylation, metabolic synthesis of tunicamycin, and the like. In addition, the derivative can contain one or more non-natural amino acids, such as using ambrx technology (see, Wolfson, Chem. Biol., 2006, 13, 1011-1012).

In some embodiments, the antibodies, or antigen-binding fragments thereof, are derivatized through glycosylation. Suitable biantennary complexes can be composed of a core structure having two N-acetylglucosamine (GlcNAc), three mannose, and two GlcNAc residues that are β-1,2 linked to α-6 mannose and α-3 mannose to form two antennae. One or more fucose (Fuc), galactose (Gal), high mannose glycans Man-5 or Man-9, bisecting GlcNAc, and sialic acid including N-acetylneuraminic acid (NANA) or N-glycolylneuraminic acid (NGNA) residues may be attached to the core. N-linked glycoforms may include GO (protein having a core biantennary glycosylation structure), GOF (fucosylated GO), GOF GlcNAc, G1 (protein having a core glycosylation structure with one galactose residue), GIF (fucosylated G1), G2 (protein having a core glycosylation structure with two galactose residues), and/or G2F (fucosylated G2). In some embodiments, an antibody has a GOF glycan.

The antibodies, or antigen-binding fragments thereof, can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and, optionally, secreted into the medium in which the host cells are cultured, from which medium the antibodies can be recovered. Standard recombinant DNA methodologies can be used to obtain antibody heavy and light chain genes, incorporate these genes into recombinant expression vectors and introduce the vectors into host cells (see, Molecular Cloning; A Laboratory Manual, Second Edition, Sambrook, Fritsch and Maniatis (eds.), Cold Spring Harbor, N.Y., 1989; Current Protocols in Molecular Biology, Ausubel et al., eds., Greene Publishing Associates, 1989; and U.S. Pat. No. 4,816,397).

In some embodiments, to generate nucleic acid molecules encoding the antibodies, or antigen-binding fragments thereof, described herein, DNA fragments encoding the light and heavy chain variable regions are first obtained. These DNAs can be obtained by amplification and modification of germline DNA or cDNA encoding light and heavy chain variable sequences, for example, using the polymerase chain reaction (PCR). Germline DNA sequences for human heavy and light chain variable region genes are known in the art (see, the “VBASE” human germline sequence database; Kabat et al., 1991, Sequences of Proteins of Immunological Interest, Fifth Edition, U.S. Department of Health and Human Services, NIH Publication No. 91-3242; Tomlinson et al., J. Mol. Biol., 1992, 22T, 116-198; and Cox et al., Eur. J. Immunol., 1994, 24, 827-836). A DNA fragment encoding the heavy or light chain variable region can be synthesized and used as a template for mutagenesis to generate a variant as described herein using routine mutagenesis techniques; alternatively, a DNA fragment encoding the variant can be directly synthesized.

These DNA fragments can be further manipulated by standard recombinant DNA techniques, for example, to convert the variable region genes to full-length antibody chain genes, to Fab fragment genes or to a scFv gene. In these manipulations, a V_{H}-or V_{L}-encoding DNA fragment is operatively linked to another DNA fragment encoding another protein, such as an antibody constant region or a flexible linker. The phrase “operatively linked,” as used in this context, is intended to mean that the two DNA fragments are joined such that the amino acid sequences encoded by the two DNA fragments remain in-frame.

The isolated DNA molecules encoding the V_{H} region can be converted to a full-length heavy chain gene by operatively linking the V_{H}-encoding DNA to another DNA molecule encoding heavy chain constant regions (CH₁, CH₂, CH₃ and, optionally, CH₄). The sequences of human heavy chain constant region genes are known in the art (see, Kabat et al., 1991, Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region. In some embodiments, the heavy chain constant region is an IgG1 or IgG4 constant region. For a Fab fragment heavy chain gene, the V_{H}-encoding DNA can be operatively linked to another DNA molecule encoding only the heavy chain CH₁ constant region.

The isolated DNA encoding the V_{L} region can be converted to a full-length light chain gene (as well as a Fab light chain gene) by operatively linking the V_{L}encoding DNA to another DNA molecule encoding the light chain constant region, C_{L} . . . . The sequences of human light chain constant region genes are known in the art (see, Kabat et al., 1991, Sequences of Proteins of Immunological Interest, Fifth Edition (U.S. Department of Health and Human Services, NIH Publication No. 91-3242)) and DNA fragments encompassing these regions can be obtained by standard PCR amplification. The light chain constant region can be a kappa or lambda constant region. In some embodiments, the light chain constant region is a kappa constant region. To create an scFv gene, the V_{H}- and V_{L}encoding DNA fragments are operatively linked to another fragment encoding a flexible linker, for example, encoding the amino acid sequence (Gly4Ser)₃, such that the V_{H} and V_{L} sequences can be expressed as a contiguous single-chain protein, with the V_{L} and V_{H} regions joined by the flexible linker (see, Bird et al., Science, 1988, 242, 423-426; Huston et al., Proc. Natl. Acad. Sci. USA, 1988, 85, 5879-5883; and McCafferty et al., Nature, 1990, 348, 552-554).

To express the antibodies, or antigen-binding fragments thereof, described herein, DNA molecules encoding partial or full-length light and heavy chains, obtained as described herein, can be inserted into expression vectors such that the genes are operatively linked to transcriptional and translational control sequences. In this context, the phrase “operatively linked” is intended to mean that an antibody gene is ligated into a vector such that transcriptional and translational control sequences within the vector serve their intended function of regulating the transcription and translation of the antibody gene. The expression vector and expression control sequences can be chosen to be compatible with the expression host cell used. The antibody light chain gene and the antibody heavy chain gene can be inserted into separate vectors or, more typically, both genes are inserted into the same expression vector.

The antibody genes can be inserted into the expression vector by standard methods such as, for example, ligation of complementary restriction sites on the antibody gene fragment and vector, or blunt end ligation if no restriction sites are present. Prior to insertion of the light or heavy chain sequences, the expression vector can already carry antibody constant region sequences. For example, one approach to converting the V_{H} and V_{L} sequences to full-length antibody genes is to insert them into expression vectors already encoding heavy chain constant and light chain constant regions, respectively, such that the V_{H} segment is operatively linked to the CH segment(s) within the vector and the V_{L} segment is operatively linked to the C_{L} segment within the vector. Additionally or alternately, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide, such as a signal peptide from a non-immunoglobulin protein.

In addition to the antibody chain genes, the recombinant expression vectors can carry regulatory sequences that control the expression of the antibody chain genes in a host cell. Such regulatory sequences are described, for example, in Goeddel, Gene Expression Technology: Methods in Enzymology 185 (Academic Press, San Diego, Calif., 1990). It will be appreciated by those skilled in the art that the design of the expression vector, including the selection of regulatory sequences may depend on such factors as the choice of the host cell to be transformed, the level of expression of protein desired, and the like. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (such as the adenovirus major late promoter (AdMLP)) and polyoma. Viral regulatory elements and sequences thereof are known in the art (see, e.g., U.S. Pat. Nos. 5,168,062, 4,510,245, and 4,968,615).

The recombinant expression vectors of the disclosure can also carry additional sequences, such as sequences that regulate replication of the vector in host cells (such as origins of replication) and selectable marker genes. The selectable marker gene facilitates selection of host cells into which the vector has been introduced (see, U.S. Pat. Nos. 4,399,216, 4,634,665, and 5,179,017). For example, typically the selectable marker gene confers resistance to drugs, such as G418, puromycin, blasticidin, hygromycin, or methotrexate, on a host cell into which the vector has been introduced. Suitable selectable marker genes include the dihydrofolate reductase (DHFR) gene (for use in DHFR host cells with methotrexate selection/amplification) and the neo gene (for G418 selection). For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, such as electroporation, lipofection, calcium-phosphate precipitation, DEAE-dextran transfection, and the like.

The antibodies, or antigen-binding fragments thereof, can be expressed in either prokaryotic or eukaryotic host cells. In some embodiments, expression of antibodies, or antigen-binding fragments thereof, can be performed in eukaryotic cells, such as mammalian host cells, for secretion of a properly folded and immunologically active antibody. Exemplary mammalian host cells for expressing the recombinant antibodies, or antigen-binding fragments thereof, of the disclosure include Chinese Hamster Ovary (CHO cells) (including DHFR CHO cells (see, Urlaub, Proc. Natl. Acad. Sci. USA, 1980, 77, 4216-4220), used with a DHFR selectable marker (see, Kaufman, Mol. Biol., 1982, 159, 601-621), NSO myeloma cells, COS cells, 293 cells, and SP2/0 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies, or antigen-binding fragments thereof, can be recovered from the culture medium using standard protein purification methods. Host cells can also be used to produce portions of intact antibodies, such as Fab fragments or scFv molecules. It is understood that variations on the above procedure are within the scope of the present disclosure. For example, it can be desirable to transfect a host cell with DNA encoding either the light chain or the heavy chain (but not both) of antibodies, or antigen-binding fragments thereof, described herein.

Recombinant DNA technology can also be used to remove some or all of the DNA encoding either or both of the light and heavy chains that is not necessary for binding to the antigen. The molecules expressed from such truncated DNA molecules are also encompassed by the antibodies, or antigen-binding fragments thereof, described herein.

In addition, bifunctional antibodies, or antigen-binding fragments thereof, can be produced in which one heavy and one light chain are an antibody of the disclosure capable of binding to a first antigen and the other heavy and light chain are specific for an antigen other than the first antigen by crosslinking an antibody of the disclosure to a second antibody by standard chemical crosslinking methods. Bifunctional antibodies can also be made by expressing a nucleic acid engineered to encode a bifunctional antibody.

For recombinant expression of the antibodies, or antigen-binding fragments thereof, described herein, the host cell can be co-transfected with two expression vectors of the disclosure, the first vector encoding a heavy chain derived polypeptide and the second vector encoding a light chain derived polypeptide. Typically, the two vectors each contain a separate selectable marker. Alternately, a single vector can be used which encodes both heavy and light chain polypeptides.

Once a nucleic acid encoding one or more portions of the antibodies, or antigen-binding fragments thereof, with desired CDR sequences is generated, further alterations can be introduced into the coding sequence, for example, to generate nucleic acids encoding antibodies with different CDR sequences, antibodies with reduced affinity to the Fc receptor, or antibodies of different subclasses.

The antibodies, or antigen-binding fragments thereof, described herein can also be produced by chemical synthesis (such as by the methods described in Solid Phase Peptide Synthesis, 2nd ed., 1984 The Pierce Chemical Co., Rockford, Ill.). Variant antibodies, or antigen-binding fragments thereof, can also be generated using a cell-free platform (see, Chu et al., Biochemia, 2001, 2).

Once antibodies, or antigen-binding fragments thereof, described herein have been produced by recombinant expression, they can be purified by any method known in the art for purification of an immunoglobulin molecule, for example, by chromatography (such as ion exchange, affinity, particularly by affinity for Protein A, Protein G or Protein L selection, and sizing column chromatography), centrifugation, differential solubility, or by any other standard technique for the purification of proteins. Further, the antibodies, or antigen-binding fragments thereof, described herein can be fused to heterologous polypeptide sequences described herein or otherwise known in the art to facilitate purification.

Once isolated, the antibodies, or antigen-binding fragments thereof, can, if desired, be further purified, such as by high performance liquid chromatography (see, Fisher, Laboratory Techniques In Biochemistry And Molecular Biology (Work and Burdon, eds., Elsevier, 1980)), or by gel filtration chromatography on a Superdex™ 75 column (Pharmacia Biotech AB, Uppsala, Sweden).

The present disclosure provides vectors comprising the nucleic acid molecules described herein. The present disclosure also provides prokaryotic host cells transformed with the vector. The present disclosure also provides eukaryotic host cells transformed with the vector. In some embodiments, the eukaryotic host cell is a mammalian host cell.

In some embodiments, the antibodies, or antigen-binding fragments thereof, are conjugated to an effector moiety. In some embodiments, the antibodies, or antigen-binding fragments thereof, are modified by the covalent attachment of any type of molecule to the antibodies, or antigen-binding fragments thereof, such that covalent attachment does not interfere with binding to an antigen. In some embodiments, the effector moiety is a detectable label, a cytotoxic agent, a chemotherapeutic agent, or a nucleic acid molecule. The effector moiety can also be an antineoplastic agent, a drug, a toxin, a biologically active protein (such as an enzyme), another antibody or antibody fragment, a synthetic or naturally occurring polymer, a nucleic acid molecule, a radionuclides (such as radioiodide), a radioisotope, a chelated metal, a nanoparticle, or a reporter group (such as a fluorescent compound or a compound which can be detected by NMR or ESR spectroscopy).

In some embodiments, the antibodies, or antigen-binding fragments thereof, can be conjugated to a cytotoxic agent, a radionuclide, or a drug moiety to modify a particular biological response. The effector moiety can be a protein or polypeptide, such as, for example, a toxin (such as abrin, ricin A, saporin, Pseudomonas exotoxin, diphtheria toxin, ethidium bromide or PE40, PE38, gelonin, RNAse, peptide nucleic acids (PNAs), ribosome inactivating protein (RIP) type-1 or type-2, pokeweed anti-viral protein (PAP), bryodin, momordin, chemotherapeutic agents, and bouganin), a signaling molecule (such as α-interferon, β-interferon, nerve growth factor, platelet derived growth factor or tissue plasminogen activator), a thrombotic agent or an anti-angiogenic agent (such as, angiostatin or endostatin) or a biological response modifier such as a cytokine or growth factor (such as, interleukin-1 (IL-1), interleukin-2 (IL-2), interleukin-6 (IL-6), granulocyte macrophage colony stimulating factor (GM-CSF), granulocyte colony stimulating factor (G-CSF), or nerve growth factor (NGF)).

In some embodiments, the cytotoxic agent is a small molecule, a prodrug, a maytansinoid, or a toxin. In some embodiments, the antibody, or antigen-binding fragment thereof, comprises from 3 to 5 maytansinoid molecules per antibody, or antigen-binding fragment thereof. In some embodiments, the maytansinoid is conjugated to the antibody, or antigen-binding fragment thereof, by a chemical linker chosen from N-succinimidyl-3-(2-pyridyldithio) propionate, N-succinimidyl-4-(2-pyridylthio)pentanoate (SPP), and succinimidyl-4-(N-maleimidomethyl)cyclohexanel-1-carboxylate. In some embodiments, the cytotoxic agent is taxol, cytochalasin B, gramicidin D, ethidium bromide, emetine, mitomycin, etoposide, tenoposide, vincristine, vinblastine, colchicin, doxorubicin, daunorabicin, dihydroxy anthracin dione, mitoxantrone, mithramycin, actinomycin D, 1-dehydrotestosterone, glucocorticoids, procaine, tetracaine, lidocaine, propranolol, or puromycin.

In some embodiments, the detectable label is a radioactive compound, a fluorescent compound, a chromophore, an enzyme, an imaging agent, a metal ion, or a substrate. In some embodiments, a fluorescent moiety includes, but is not limited to, fluorescein, fluorescein isothiocyanate, rhodamine, 5-dimethylamine-1-napthalenesulfonyl chloride, phycoerythrin, and the like. Useful enzymatic labels include, but are not limited to, alkaline phosphatase, horseradish peroxidase, glucose oxidase, and the like.

In some embodiments, the effector moiety is an antimetabolite (such as, methotrexate, 6-mercaptopurine, 6-thioguanine, cytarabine, and 5-fluorouracil decarbazine), an alkylating agent (such as, mechlorethamine, thioepa chlorambucil, melphalan, carmustine (BSNU) and lomustine (CCNU), cyclothosphamide, busulfan, dibromomannitol, streptozotocin, mitomycin C5 and cisdichlorodiamine platinum (II) (DDP) cisplatin), an anthracycline (such as, daunorubicin (formerly daunomycin) and doxorubicin), an antibiotic (such as, dactinomycin (formerly actinomycin), bleomycin, mithramycin, anthramycin (AMC), calicheamicins or duocarmycins), or an anti-mitotic agent (such as, vincristine and vinblastine).

In some embodiments, the radionuclides is, but is not limited to, ¹³N, ¹⁸F, ³²P, ⁶⁴Cu, ⁶⁶Ga, ⁶⁷Ga, ⁶⁸Ga, ⁶⁷Cu, ⁷⁷Br, ^{80m}Br, ⁸²Rb, ⁸⁶Y, ⁹⁰Y, ⁹⁵Ru, ⁹⁷Ru, ^{99m}Tc, ¹⁰³Ru, ¹⁰⁵Ru, ¹¹¹In, ^{113m}In, ¹¹³Sn, ^{121m}Te, ^{122m}Te, ^{125m}Te, ¹²³I, ¹²⁴I, ¹²⁵I, ¹²⁶I, ¹³¹I, ¹³³I, ¹⁶⁵Tm, ¹⁶⁷Tm, ¹⁶⁸Tm, ¹⁷⁷Lu, ¹⁸⁶Re, ¹⁸⁸Re, ^{195m}Hg, ²¹¹At, ²¹²Bi, ²¹³Bi, and ²²⁵Ac.

In some embodiments, the chemotherapeutic agent is cisplatin, carboplatin, oxaliplatin, nedaplatin, triplatin tetranitrate, phenanthriplatin, picoplatin, satraplatin, methotrexate, vincristine, doxorubicin, tunicamycin, oligomycin, bortezomib, MG132, 5-flurouracil, sorafenib, flavopiridol, gemcitabine, taxol, mercaptopurine, thioguanine, hydroxyurea, cytarabine, mitomycin, cyclophosphamide, ifosfamide, nitrosourea, dacarbazine, procarbizine, an etoposide, a campathecin, bleomycin, idarubicin, daunorubicin, dactinomycin, distamycin A, etidium, netropsin, auristatin, amsacrine, prodigiosin, bortexomib, pibenzimol, tomaymycin, duocarmycin SA, plicamycin, mitoxantrone, asparaginase, vinblastine, vinorelbine, paclitaxel, docetaxel, CPT-11, gleevec, erlotinib, gefitinib, ibrutinib, crizotinib, ceritinib, lapatinib, navitoclax, or regorafenib.

In some embodiments, the nucleic acid molecule is a single layer nucleic acid carrier, a 1.5 layer nucleic acid carrier, a two layer nucleic acid carrier, a 2.5 layer nucleic acid carrier, or a three layer nucleic acid carrier (such as those disclosed in, for example, PCT Publications WO 17/143156 and WO 17/143171).

Techniques for conjugating such effector moieties to antibodies are well known in the art (see, Hellstrom et al., Controlled Drug Delivery, 2nd Ed., at pages 623-53 (Robinson et al., eds., 1987)); Thorpe et al., Immunol. Rev., 1982, 62, 119-58; and Dubowchik et al., Pharmacology and Therapeutics, 1999, 83, 67-123).

In some embodiments, the antibodies, or antigen-binding fragments thereof, can be fused via a covalent bond (such as, a peptide bond), through the antibody's N-terminus or C-terminus or internally, to an amino acid sequence of another protein (or portion thereof; for example, at least a 10, 20 or 50 amino acid portion of the protein). The antibodies, or antigen-binding fragments thereof, can be linked to the other protein at the N-terminus of the constant domain of the antibody. Recombinant DNA procedures can be used to create such fusions, for example, as described in PCT Publication WO 86/01533 and European Patent EP0392745. In some embodiments, the effector moiety can increase half-life in vivo, and/or enhance the delivery of an antibody across an epithelial barrier to the immune system. Examples of suitable effector moieties of this type include polymers, albumin, albumin-binding proteins or albumin-binding compounds such as those described in PCT Publication WO 2005/117984.

In some embodiments, the antibodies, or antigen-binding fragments thereof, can be conjugated to a small molecule toxin. In some embodiments, the antibodies, or antigen-binding fragments thereof, can be conjugated to a dolostatin or a dolostatin peptidic analog or derivative, such as an auristatin (see, U.S. Pat. Nos. 5,635,483 and 5,780,588). The dolastatin or auristatin drug moiety may be attached to the antibody through its N-terminus, C-terminus or internally (see, PCT Publication WO 02/088172). Exemplary auristatin embodiments include the N-terminus linked monomethylauristatin drug moieties DE and DF, as disclosed in U.S. Pat. No. 7,498,298 (disclosing linkers and methods of preparing monomethylvaline compounds such as MMAE and MMAF conjugated to linkers).

Antibodies, or antigen-binding fragments thereof, can also be conjugated to liposomes for targeted delivery (see, Park et al., Adv. Pharmacol., 1997, 40, 399-435; and Marty et al., Methods Molec. Med., 2004, 109,389-401).

In some embodiments, the antibodies, or antigen-binding fragments thereof, can be attached to PEG moieties. In some embodiments, the antibodies, or antigen-binding fragments thereof, and the PEG moieties can be attached through any available amino acid side-chain or terminal amino acid functional group located in the antibodies, or antigen-binding fragments thereof, for example, any free amino, imino, thiol, hydroxyl or carboxyl group. Such amino acids can occur naturally in the antibodies, or antigen-binding fragments thereof, or can be engineered into the fragment using recombinant DNA methods (see, U.S. Pat. No. 5,219,996). Multiple sites can be used to attach two or more PEG moieties. PEG moieties can be covalently linked through a thiol group of at least one cysteine residue located in the antibodies, or antigen-binding fragments thereof. Where a thiol group is used as the point of attachment, appropriately activated effector moieties, for example, thiol selective derivatives such as maleimides and cysteine derivatives, can be used.

In some embodiments, the antibodies, or antigen-binding fragments thereof, can comprise a modified Fab′ fragment which is PEGylated. The PEG moiety can be attached to a cysteine in the hinge region. In some embodiments, a PEG-modified Fab′ fragment has a maleimide group covalently linked to a single thiol group in a modified hinge region. A lysine residue can be covalently linked to the maleimide group and to each of the amine groups on the lysine residue can be attached a methoxypoly(ethyleneglycol) polymer having a molecular weight of approximately 20,000 Da. The total molecular weight of the PEG attached to the Fab′ fragment can therefore be approximately 40,000 Da.

In some embodiments, the antigen-binding fragment can be an Fab, an F(ab′)₂, an Fv, an scFv, an scFv-Fc, a diabody, or a minibody fragment. In some embodiments, the antigen-binding fragment can be an Fab fragment. In some embodiments, the antigen-binding fragment can be an F(ab′)₂ fragment. In some embodiments, the antigen-binding fragment can be an Fv fragment. In some embodiments, the antigen-binding fragment can be an scFv fragment. In some embodiments, the antigen-binding fragment can be an scFv-Fc fragment. In some embodiments, the antigen-binding fragment can be a diabody fragment. In some embodiments, the antigen-binding fragment can be a minibody fragment.

In some embodiments, the antibody, or antigen-binding fragment thereof, is a therapeutic antibody. In some embodiments, the antibody, or antigen-binding fragment thereof, comprises 3F8, 8H9, abagovomab, abciximab, abituzumab, abrezekimab, abrilumab, actoxumab, adalimumab, adecatumumab, aducanumab, afasevikumab, afelimomab, alacizumab pegol, alemtuzumab, alirocumab, altumomab pentetate, amatuximab, amivantamab, anatumomab mafenatox, andecaliximab, anetumab ravtansine, anifrolumab, ansuvimab, anrukinzumab, apolizumab, aprutumab ixadotin, arcitumomab, ascrinvacumab, aselizumab, atezolizumab, atidortoxumab, atinumab, atoltivimab, maftivimab, odesivimab, atorolimumab, avelumab, azintuxizumab vedotin, bamlanivimab, bapineuzumab, basiliximab, bavituximab, BCD-100, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimekizumab, birtamimab, bivatuzumab, bleselumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, casirivimab, capromab, carlumab, carotuximab, catumaxomab, a cBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofetuzumab pelidotin, coltuximab ravtansine, conatumumab, concizumab, cosfroviximab, crenezumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, dinutuximab beta, diridavumab, domagrozumab, dorlimomab aritox, dostarlimab, drozitumab, DS-8201, duligotuzumab, dupilumab, durvalumab, dusigitumab, duvortuxizumab, ecromeximab, eculizumab, edobacomab, edrecolomab, efalizumab, efungumab, eldelumab, elezanumab, elgemtumab, elotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epcoritamab, epitumomab cituxetan, epratuzumab, eptinezumab, erenumab, erlizumab, ertumaxomab, etaracizumab, etesevimab, etigilimab, etrolizumab, evinacumab, evolocumab, exbivirumab, fanolesomab, faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab, foralumab, foravirumab, fremanezumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imdevimab, imgatuzumab, inclacumab, indatuximab ravtansine, indusatumab vedotin, inebilizumab, infliximab, intetumumab, inolimomab, inotuzumab ozogamicin, ipilimumab, Iomab-B, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, loncastuximab tesirine, losatuxizumab vedotin, lilotomab satetraxetan, lintuzumab, lirilumab, lodelcizumab, lokivetmab, lorvotuzumab mertansine, lucatumumab, lulizumab pegol, lumiliximab, lumretuzumab, lupartumab, lupartumab amadotin, lutikizumab, maftivimab, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxetumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odesivimab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, prezalumab, plozalizumab, pogalizumab, polatuzumab vedotin, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab, raxibacumab, ravagalimab, ravulizumab, refanezumab, regavirumab, regdanvimab, relatlimab, remtolumab, reslizumab, rilotumumab, rinucumab, risankizumab, rituximab, rivabazumab pegol, robatumumab, SII RMAb, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxetan, tadocizumab, tafasitamab, talacotuzumab, talizumab, talquetamab, tamtuvetmab, tanezumab, taplitumomab paptox, tarextumab, tavolimab, teclistamab, tefibazumab, telimomab aritox, telisotuzumab, telisotuzumab vedotin, tenatumomab, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab duocarmazine, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, or zolimomab aritox. In some particularly preferred embodiments, the therapeutic antibody comprises ipilimumab, nivolumab, pembrolizumab, atezolizumab, avelumab, durvalumab, cemiplimab, spartalizumab, or atezolizumab. In some embodiments, the therapeutic antibody comprises pembrolizumab. In some embodiments, the therapeutic antibody comprises comprises bersanlimab or enlimomab pegol.

The present disclosure also provides pharmaceutical compositions comprising the antibodies, or antigen-binding fragments thereof, described herein and a pharmaceutically acceptable carrier. In some embodiments, the pharmaceutical compositions further comprise a tonicity agent, a surfactant, a preservative, and/or a buffer system having a pH of about 4.0 to about 8.0. In some embodiments, the pharmaceutical compositions further comprise one or more additional therapeutic agents, such as the combination therapeutic agents described herein. In some embodiments, the pharmaceutical composition is a liquid pharmaceutical composition.

In some embodiments, the pharmaceutical compositions can be presented in unit dose forms containing a predetermined amount of an antibody, or antigen-binding fragment thereof, described herein per dose. Pharmaceutically acceptable carriers for use in the pharmaceutical compositions can take a wide variety of forms depending on the condition to be treated or route of administration.

Pharmaceutical compositions comprising the antibodies, or antigen-binding fragments thereof, described herein can be prepared for storage as lyophilized formulations or aqueous solutions by mixing the antibodies, or antigen-binding fragments thereof, having the desired degree of purity with optional pharmaceutically acceptable carriers, excipients or stabilizers typically employed in the art (all of which are referred to herein as “carriers”) such as, for example, buffering agents, stabilizing agents, preservatives, isotonifiers, non-ionic detergents, antioxidants, and other miscellaneous additives.

Buffering agents help to maintain the pH in the range that approximates physiological conditions. They can be present at concentrations ranging from about 2 mM to about 50 mM. Suitable buffering agents for use in the pharmaceutical compositions described herein can include both organic and inorganic acids and salts thereof, such as citrate buffers (such as, monosodium citrate-disodium citrate mixture, citric acid-trisodium citrate mixture, citric acid-monosodium citrate mixture, and the like), succinate buffers (such as, succinic acid monosodium succinate mixture, succinic acid-sodium hydroxide mixture, succinic acid-disodium succinate mixture, and the like), tartrate buffers (such as, tartaric acid-sodium tartrate mixture, tartaric acid-potassium tartrate mixture, tartaric acid-sodium hydroxide mixture, and the like), fumarate buffers (such as, fumaric acid-monosodium fumarate mixture, fumaric acid-disodium fumarate mixture, monosodium fumarate-disodium fumarate mixture, and the like), gluconate buffers (such as, gluconic acid-sodium glyconate mixture, gluconic acid-sodium hydroxide mixture, gluconic acid-potassium glyconate mixture, and the like), oxalate buffer (such as, oxalic acid-sodium oxalate mixture, oxalic acid-sodium hydroxide mixture, oxalic acid-potassium oxalate mixture, and the like), lactate buffers (such as, lactic acid-sodium lactate mixture, lactic acid-sodium hydroxide mixture, lactic acid-potassium lactate mixture, and the like) and acetate buffers (such as, acetic acid-sodium acetate mixture, acetic acid-sodium hydroxide mixture, and the like). Additionally, phosphate buffers, histidine buffers, and trimethylamine salts, such as Tris, can be used.

Preservatives can be added to the pharmaceutical compositions to decrease microbial growth, and can be added in amounts ranging from about 0.2% to about 1% (w/v). Suitable preservatives for use with the pharmaceutical compositions described herein include, but are not limited to, phenol, benzyl alcohol, meta-cresol, methyl paraben, propyl paraben, octadecyldimethylbenzyl ammonium chloride, benzalconium halides (such as, chloride, bromide, and iodide), hexamethonium chloride, and alkyl parabens such as methyl or propyl paraben, catechol, resorcinol, cyclohexanol, and 3-pentanol.

Isotonicifiers sometimes known as “stabilizers,” can be added to ensure isotonicity of liquid compositions and include polyhydric sugar alcohols, for example, trihydric or higher sugar alcohols, such as glycerin, erythritol, arabitol, xylitol, sorbitol, and mannitol. Stabilizers refer to a broad category of excipients, which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinositol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (such as, peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccharides such as raffinose; and polysaccharides such as dextran. Stabilizers can be present in the range from about 0.1 to about 10,000 weights per part of weight active protein.

Non-ionic surfactants or detergents (also known as “wetting agents”) can also be added to pharmaceutical compositions to help solubilize the antibodies, or antigen-binding fragments thereof, as well as to protect the antibodies, or antigen-binding fragments thereof, described herein against agitation-induced aggregation, which also permits the formulation to be exposed to shear surface stressed without causing denaturation of the proteins. Suitable non-ionic surfactants include polysorbates (20, 80, etc.), polyoxamers (184, 188 etc.), Pluronic polyols, polyoxyethylene sorbitan monoethers (TWEEN™-20, TWEEN™-80, and the like). Nonionic surfactants can be present in a range of about 0.05 mg/mL to about 1.0 mg/mL, for example, about 0.07 mg/mL to about 0.2 mg/mL.

Additional excipients, such as, bulking agents (such as, starch), chelating agents (such as, EDTA), antioxidants (such as, ascorbic acid, methionine, and vitamin E), and cosolvents can also be added to the pharmaceutical compositions.

The present disclosure also provides pharmaceutical kits containing the antibodies, or antigen-binding fragments thereof, including antibody conjugates, described herein. The pharmaceutical kits can be a package comprising the antibodies, or antigen-binding fragments thereof, described herein (such as, either in lyophilized form or as an aqueous solution) and one or more of the following: a combination therapeutic agent, a device for administering the antibodies, or antigen-binding fragments thereof, such as a pen, needle and/or syringe; and pharmaceutical grade water or buffer to re-suspend the antibodies, or antigen-binding fragments thereof, if the antibody is in lyophilized form.

In some embodiments, each unit dose of the antibodies, or antigen-binding fragments thereof, is packaged separately, and a kit can contain one or more unit doses (such as, two unit doses, three unit doses, four unit doses, five unit doses, eight unit doses, ten unit doses, or more). In some embodiments, the one or more unit doses are each housed in a syringe or pen.

Diagnostic kits containing the antibodies, or antigen-binding fragments thereof, (including antibody conjugates), described herein are also encompassed herein. The diagnostic kit can be a package comprising the antibodies, or antigen-binding fragments thereof, described herein (such as, either in lyophilized form or as an aqueous solution) and one or more reagents useful for performing a diagnostic assay. Where the antibodies, or antigen-binding fragments thereof, are labeled with an enzyme, the kits can include substrates and cofactors required by the enzyme (such as, a substrate precursor which provides the detectable chromophore or fluorophore). In addition, other additives can be included, such as stabilizers, buffers (such as, a block buffer or lysis buffer), and the like. In some embodiments, the antibodies, or antigen-binding fragments thereof, included in a diagnostic kit can be immobilized on a solid surface, or a solid surface (such as, a slide or plate) on which the antibodies, or antigen-binding fragments thereof, can be immobilized is included in the kit. The relative amounts of the various reagents can be varied to provide for concentrations in the solution of the reagents which substantially optimize the sensitivity of the assay. In some embodiments, the antibodies, or antigen-binding fragments thereof, and one or more reagents can be provided (individually or combined) as dry powders, usually lyophilized, including excipients which on dissolution will provide a reagent solution having the appropriate concentration.

The present disclosure also provides methods of detecting a cell expressing an antigen, the methods comprising contacting the cell with the antibodies, or antigen-binding fragments thereof, described herein, and detecting the antibodies, or antigen-binding fragments thereof. In some embodiments, the cell is present in a biological sample obtained from a human and the cell is contacted with the antibody, or antigen-binding fragment thereof, in vitro. In some embodiments, the cell is present in a human and the cell is contacted with the antibody, or antigen-binding fragment thereof, in vivo.

In some embodiments, the antibodies, or antigen-binding fragments thereof, described herein have a high binding affinity for an antigen. In some embodiments, the antibodies, or antigen-binding fragments thereof, have particular association rate constants (kₒₙ or k_{A} values), dissociation rate constants (k_{off} or k_{D} values), affinity constants (K_{A} values), dissociation constants (K_{D} values) and/or IC₅₀ values.

In some embodiments, the antibodies, or antigen-binding fragments thereof, bind to an antigen with a K_{A} (Kₒₙ/K_{off}) of at least about 10¹⁰ M⁻¹, at least about 4×10¹¹ M⁻¹, at least about 10¹¹ M⁻¹, at least about 4×10¹² M⁻¹, at least about 10¹² M⁻¹, at least about 4×10¹³ M⁻¹, at least about 10¹³ M⁻¹, at least about 4×10¹⁴ M⁻¹, at least about 10¹⁴ M⁻¹, at least about 4×10¹⁵ M⁻¹, or at least about 10¹⁵ M⁻¹, or with a K_{A} of any range from and to any pair of the foregoing values (such as, from about 4×10¹¹ M⁻¹ to about 4×10¹³ M⁻¹ or from about 4×10¹² M⁻¹ to about 4×10¹⁵ M⁻¹).

In some embodiments, the antibodies, or antigen-binding fragments thereof, bind to an antigen with a K_{D} (k_{off}/kₒₙ) of about 10⁻¹⁰ or less, about 4×10⁻¹¹ M or less, about 10⁻¹¹ M or less, about 4×10⁻¹² M or less, about 10⁻¹² M or less, about 4×10¹³ M or less, about 10⁻¹³ M or less, about 4×10¹⁴ M or less, about 10⁻¹⁴ M or less, about 4×10⁻¹⁵ M or less, or about 10⁻¹⁵ M or less, or with a K_{D} of any range from and to any pair of the foregoing values (such as, from about 4×10⁻¹¹ M to about 4×10⁻¹³ M or from about 4×10⁻¹² M to about 4×10⁻¹⁵ M).

In some embodiments, the K_{D} (k_{off}/kₒₙ) value is determined by assays well known in the art, such as ELISA, isothermal titration calorimetry (ITC), fluorescent polarization assay or any other biosensors such as BIAcore.

In some embodiments, the antibodies, or antigen-binding fragments thereof, bind to an antigen and inhibit the binding of the antigen's ligand at an IC₅₀ of less than about 0.02 nM, less than about 0.01 nM, less than about 0.005 nM, less than about 0.002 nM, less than about 0.001 nM, less than about 5×10⁻⁴ nM, less than about 2×10⁻⁴ nM, less than about 1×10⁻⁴ nM, less than about 5×10⁻⁵ nM, less than about 2×10⁻⁵ nM, less than about 1×10⁻⁴ nM, less than about 5×10⁻⁶ nM, less than about 2×10⁻⁶ nM, less than about 1×10⁶ nM, less than about 5×10⁻⁷ nM, less than about 2×10⁻⁷ nM, or less than about 1×10⁻⁷ nM, or with an IC₅₀ of any range from and to any pair of the foregoing values (such as, from about 0.02 nM to about 2×10⁻⁵ nM, or from about 5×10⁻⁵ nM to about 1×10⁻⁷ nM). The IC₅₀ can be measured according to methods well known in the art, such as ELISA.

The antibodies, or antigen-binding fragments thereof, including those antibodies that have been modified, such as by biotinylation, horseradish peroxidase, or any other detectable moiety (including those described above), can be used for diagnostic purposes.

In some embodiments, the antibodies, or antigen-binding fragments thereof, can be used to purify or detect an antigen, including both in vitro and in vivo diagnostic methods. For example, the antibodies, or antigen-binding fragments thereof, can be used in immunoassays for qualitatively and quantitatively measuring levels of an antigen in biological samples, or to identify the location, quantity, behavior and/or the like of an antigen in an animal. For example, measuring levels of antigen using the antibodies, or antigen-binding fragments thereof, described herein can be used to, for example: 1) diagnose or determine an increased risk of developing a cancer in a patient, 2) determine the prognosis of a patient, including the stage and grade of a tumor (particularly whether the cancer is metastatic or likely to be metastatic) and/or its potential sensitivity to antibody therapy, 3) determine the origin of a tumor, and/or 4) determine the efficacy of a treatment of a patient.

In some embodiments, the antibodies, or antigen-binding fragments thereof, can be used, for example, in conjunction with compound screening assays, for the evaluation of the effect of pharmaceutical agents on the expression and/or activity of the antigen. Additionally, the antibodies, or antigen-binding fragments thereof, can be used in conjunction with gene therapy techniques to, for example, evaluate the success of transfection of normal and/or engineered antigen-expression.

The present disclosure also provides methods of diagnosis of a neurological disease comprising detecting the amount or activity of an antigen expressed in neural tissue or in any tissue associated with a non-CNS target organ, such as the lung, liver, kidney, spleen, and the like. The diagnostic methods can employ the antibodies, or antigen-binding fragments thereof, conjugated to a diagnostic agent. The antibodies, or antigen-binding fragments thereof, can be used diagnostically, for example, to detect expression of an antigen in particular cells, tissues, or serum; or to monitor the development or progression of an immunologic response as part of a clinical testing procedure to, for example, determine the efficacy of a particular treatment regimen. Detection can be facilitated by coupling the antibodies, or antigen-binding fragments thereof, to a detectable substance. Examples of detectable substances include, but are not limited to, various enzymes, prosthetic groups, fluorescent materials (such as, fluorescein and rhodamine and their derivatives), luminescent materials, bioluminescent materials, optical agents (such as, derivatives of porphyrins, anthraquinones, anthrapyrazoles, perylenequinones, xanthenes, cyanines, acridines, phenoxazines and phenothiazines), radioactive materials, positron emitting metals using various positron emission tomographies, and nonradioactive paramagnetic metal ions (such as, Gd(III), Eu(III), Dy(III), Pr(III), Pa(IV), Mn(II), Cr(III), Co(III), Fe(III), Cu(II), Ni(II), Ti(III), and V(IV)). The detectable substance can be coupled or conjugated either directly to the antibodies, or antigen-binding fragments thereof, or indirectly, through an intermediate (such as, for example, a linker known in the art) using techniques known in the art. Examples of enzymatic labels include luciferases (such as, firefly luciferase and bacterial luciferase; see, U.S. U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, acetylcholinesterase, glucoamylase, lysozyme, saccharide oxidases (such as, glucose oxidase, galactose oxidase, and glucose-6-phosphate dehydrogenase), heterocyclic oxidases (such as, uricase and xanthine oxidase), lactoperoxidase, microperoxidase, and the like. Examples of suitable prosthetic group complexes include, but are not limited to, streptavidin/biotin and avidin/biotin; examples of suitable fluorescent materials include, but are not limited to, umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include, but are not limited to, luciferase, luciferin, and aequorin; and examples of suitable radioactive material include, but are not limited to, ¹²⁵I, ¹³¹I, ¹¹¹In or ⁹⁹Tc.

The disclosure also provides methods for detecting expression of an antigen on a cell, comprising contacting a biological sample from a patient using one or more of the antibodies, or antigen-binding fragments thereof, described herein (optionally conjugated to detectable moiety), and detecting whether or not the sample is positive for an antigen expression, or whether the sample has altered (such as, reduced or increased) expression as compared to a control sample. The biological sample may include biopsies of various tissues including, without limitation: skin, muscle, breast, prostate, cervical, ovarian, brain, testicular, gastrointestinal, ocular, liver, kidney, and pulmonary. Cellular examples of biological samples include tumor cells, skin cells, muscle cells, blood cells, ovarian cells, brain cells, prostate cells, breast cells, testicular cells, gastrointestinal cells, ocular cells, liver cells, kidney cells, cervical cells, and lung cells. The biological sample may also be a biological fluid.

The presence of antigen-expressing cells in a biological sample can be indicative of the presence of cancer and may be indicative of metastases, particularly when present in quantities greater than that of normal healthy subjects. The loss of antigen-expressing cells in a patient, particularly one undergoing treatment, over time can be indicative of remission (i.e., successful treatment), while the lack of change in antigen-expressing cell levels in a patient undergoing treatment can be indicative of resistance to the therapy and indicates that a different therapeutic strategy could be employed. Similarly, the gain of antigen-expressing cells in a patient over time can be indicative of recurrence. Additionally, the imaging techniques described herein may be employed to monitor the size of the tumor to determine the efficacy of a treatment. In some embodiments, other cancer diagnostic assays can be performed to confirm the results obtained with the methods described herein.

In some embodiments, a biological sample (such as, a tumor sample) can be obtained from a subject and the presence of antigen-expressing cells determined. The number of antigen-expressing cells can be correlated with tumor grade. In some embodiments, the number of antigen-expressing cells in the biological sample is compared to the number of antigen-expressing cells in a corresponding biological sample from a healthy individual to determine the modulation of antigen-expressing cells in the tumor. Subjects comprising the tumor can be treated with pharmaceutical agents to modulate the activity of antigen-expressing cells to normal, healthy levels.

Diseases that can be diagnosed using the present methods include, but are not limited to, neurological cancers such as primary brain tumors including glioma (glioblastoma), meningioma, neurinoma, pituitary adenoma, medulloblastoma, craniopharyngioma, hemangioma, epidermoid, sarcoma and intracranial metastasis from other tumor sources. In some embodiments, the antibodies, or antigen-binding fragments thereof, described herein can be used to diagnose glioblastoma multiforme (GBM).

The present disclosure also provides methods of treating a cancer expressing antigen, the methods comprising administering to a human patient in need thereof the antibodies, or antigen-binding fragments thereof, described herein. In some embodiments, the methods involve administering to a human patient having a solid tumor an amount of the antibodies, or antigen-binding fragments thereof, described herein to provide therapeutic benefit.

In some embodiments, the antibodies, or antigen-binding fragments thereof, described herein can be administered to a patient by a variety of routes such as orally, transdermally, subcutaneously, intranasally, intravenously, intraarterially, intramuscularly, intraocularly, topically, locally, intrathecally, intracerebroventricularly, intraspinally, and inracranially. The most suitable route for administration in any given case will depend on the particular antibody, the subject, and the nature and severity of the disease and the physical condition of the subject. In some embodiments, the antibodies, or antigen-binding fragments thereof, can be formulated as an aqueous solution. In some embodiments, the antibodies, or antigen-binding fragments thereof, are administered intravenously or intracranially.

The antibodies, or antigen-binding fragments thereof, described herein can be used to treat various antigen-expressing neoplasms. In some embodiments, the antibodies, or antigen-binding fragments thereof, described herein can be used to treat antigen-expressing cancers, such as sarcomas with properties of skeletal muscle, and skin tumors. In some embodiments, the antibodies, or antigen-binding fragments thereof, described herein can be used to treat antigen-expressing neurological cancers, such as a brain tumor or a small brain lesion, such as micrometastases, in a patient. In some embodiments, the antigen-expressing cancer is a neurological cancer. In some embodiments, the neurological cancer is a primary brain tumor, a glioblastoma, a glioma, a meningioma, a neurinoma, a pituitary adenoma, a medulloblastoma, a craniopharyngioma, a hemangioma, an epidermoid, a sarcoma, or an intracranial metastasis from other tumor sources. In some embodiments, the neurological cancer is a glioblastoma. In some embodiments, the glioblastoma is glioblastoma multiforme (GBM).

In some embodiments, the antigen-expressing brain tumor is a GBM tumor containing GBM tumor-initiating cells. In some embodiments, treatment with the antibodies, or antigen-binding fragments thereof, described herein results in the inhibition of the proliferation of GBM tumor-initiating cells. In some embodiments, the antibodies, or antigen-binding fragments thereof, further inhibit self-renewal of GBM tumor-initiating cells. Inhibition of cell proliferation and/or self-renewal may lead to improvement in the signs or symptoms of disease. For example, such therapy may result in an improvement in survival (overall survival and/or progression free survival) and/or may result in an objective clinical response (partial or complete). In some embodiments, the antibodies, or antigen-binding fragments thereof, are internalized by the GBM tumor cell, resulting in the increased therapeutic efficacy of the antibodies, or antigen-binding fragments thereof, in killing the GBM tumor cell to which it binds. In some embodiments, the antibodies, or antigen-binding fragments thereof, act as antagonists of antigen biological activity, and can additionally be used as a method for the inhibition of abnormal antigen activity.

In some embodiments, the antibodies, or antigen-binding fragments thereof, are useful in the treatment of non-neurological antigen-expressing tumors, including cancers and benign tumors. Cancers that are amenable to treatment by the antibodies, or antigen-binding fragments thereof, described herein include those that overexpress the antigen. In some embodiments, cancers that are amenable to treatment by the antibodies, or antigen-binding fragments thereof, described herein include epithelial cell cancers. In some embodiments, cancers that are amenable to treatment by the antibodies, or antigen-binding fragments thereof, described herein include, but are not limited to, breast cancer, ovarian cancer, lung cancer, colorectal cancer, anal cancer, prostate cancer, kidney cancer, bladder cancer, head and neck cancer, ovarian cancer, pancreatic cancer, skin cancer, oral cancer, esophageal cancer, vaginal cancer, cervical cancer, cancer of the spleen, testicular cancer, cancer of the thymus, head and neck cancer, and colorectal cancer. The cancer may be newly diagnosed and naive to treatment, or may be relapsed, refractory, or relapsed and refractory, or a metastatic form of a solid tumor.

In some embodiments, the antibodies, or antigen-binding fragments thereof, described herein can be used in the treatment of an antigen-expressing blood malignancy, including, but not limited to, myelomas (such as, multiple myeloma), lymphomas (such as, Hodgkin's lymphoma, non-Hodgkin's lymphoma, Waldenström's macroglobulinemia, and mantle cell lymphoma), leukemias (such as, chronic lymphocytic leukemia, acute myeloid leukemia, and acute lymphocytic leukemia), and myelodysplastic syndromes. In some embodiments, the methods comprise administering to a human patient having a blood malignancy the antibodies, or antigen-binding fragments thereof, described herein to provide therapeutic benefit.

In some embodiments, the administration of the antibodies, or antigen-binding fragments thereof, described herein is repeated after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens, a patient can receive antibody therapy for a prolonged period of time, such as 6 months, 1 year, or more. The amount of the antibodies, or antigen-binding fragments thereof, described herein administered to the patient is a therapeutically effective amount. As used herein, a “therapeutically effective” amount of the antibodies, or antigen-binding fragments thereof, described herein can be administered as a single dose or over the course of a therapeutic regimen, such as over the course of a week, two weeks, three weeks, one month, three months, six months, one year, or longer. Exemplary therapeutic regimens are further described herein. Treatment of a disease encompasses the treatment of patients already diagnosed as having any form of the disease at any clinical stage or manifestation; the delay of the onset or evolution or aggravation or deterioration of the symptoms or signs of the disease; and/or preventing and/or reducing the severity of the disease.

The use of the antibodies, or antigen-binding fragments thereof, described herein to treat cancer in a patient can result in any demonstrated clinical benefit compared with no therapy (when appropriate) or to a known standard of care. Clinical benefit can be assessed by any method known to one of ordinary skill in the art. In some embodiments, clinical benefit is assessed based on objective response rate (ORR) (determined using RECIST version 1.1), duration of response (DOR), progression-free survival (PFS), and/or overall survival (OS). In some embodiments, a complete response indicates therapeutic benefit. In some embodiments, a partial response indicates therapeutic benefit. In some embodiments, stable disease indicates therapeutic benefit. In some embodiments, an increase in overall survival indicates therapeutic benefit. In some embodiments, therapeutic benefit may constitute an improvement in time to disease progression and/or an improvement in symptoms or quality of life. In some embodiments, a therapeutic benefit may not translate to an increased period of disease control, but rather a markedly reduced symptom burden resulting in improved quality of life. As will be apparent to those of skill in the art, a therapeutic benefit may be observed using the antibodies, or antigen-binding fragments thereof, described herein alone (monotherapy) or adjunctive to, or with, other anti-cancer therapies and/or targeted or non-targeted anti-cancer agents.

In some embodiments, a therapeutic benefit can be assessed using standard clinical tests designed to measure the response to a new treatment for cancer. To assess the therapeutic benefits of the antibodies, or antigen-binding fragments thereof, described herein one or a combination of the following tests can be used: 1) the Response Evaluation Criteria In Solid Tumors (RECIST) version 1.1; 2) immune-related RECIST (irRECIST); 3) the Eastern Cooperative Oncology Group (ECOG) Performance Status; 4) immune-related response criteria (irRC); 5) disease evaluable by assessment of tumor antigens; 6) validated patient reported outcome scales; and/or 7) Kaplan-Meier estimates for overall survival and progression free survival.

The present disclosure also provides combination therapy methods comprising administering at least two agents to a patient, the first of which is an antibody, or antigen-binding fragment thereof, described herein, and the second of which is a combination therapeutic agent. The antibodies, or antigen-binding fragments thereof, described herein and the combination therapeutic agent can be administered simultaneously, sequentially, or separately. The combinatorial therapy methods can result in an additive or a greater than additive effect.

In the present methods, the antibodies, or antigen-binding fragments thereof, described herein and the combination therapeutic agent can be administered concurrently, either simultaneously or successively. The antibodies, or antigen-binding fragments thereof, described herein and the combination therapeutic agent are administered successively if they are administered to the patient on the same day, for example, during the same patient visit. Successive administration can occur 1, 2, 3, 4, 5, 6, 7 or 8 hours apart. In contrast, the antibodies, or antigen-binding fragments thereof, described herein and the combination therapeutic agent are administered separately if they are administered to the patient on different days, for example, the antibodies, or antigen-binding fragments thereof, and the combination therapeutic agent can be administered at a 1-day, 2-day or 3-day, one-week, 2-week or monthly intervals. In the methods of the present disclosure, administration of the antibodies, or antigen-binding fragments thereof, described herein can precede or follow administration of the combination therapeutic agent. In some embodiments, the antibodies, or antigen-binding fragments thereof, described herein and combination therapeutic agent can be administered concurrently for a period of time, followed by a second period of time in which the administration of the antibodies, or antigen-binding fragments thereof, and the combination therapeutic agent is alternated.

In some embodiments, the combination therapeutic agent is a chemotherapeutic agent, an anti-angiogenic agent, an anti-rheumatic drug, an anti-inflammatory agent, a radiotherapeutic, an immunosuppressive agent, or a cytotoxic drug. The antibodies, or antigen-binding fragments thereof, described herein can be used in combination with conventional cancer therapies, such as surgery, radiotherapy, chemotherapy, or combinations thereof.

In some embodiments, the methods further comprise one or both of surgically resecting tumor cells and/or administering radiation therapy. In some embodiments, other therapeutic agents useful for combination tumor therapy with the antibodies, or antigen-binding fragments thereof, described herein include antagonists, such as, antibodies, of other factors that are involved in tumor growth, such as HER2, HER3, HER4, VEGF, or TNF-α. In some embodiments, for treatment of cancers it may be beneficial to also administer one or more cytokines to the patient. In some embodiments, the antibodies, or antigen-binding fragments thereof, described herein is co-administered with a growth inhibitory agent.

For treatment of cancers, anti-inflammatory agents can suitably be used in combination with the antibodies, or antigen-binding fragments thereof, described herein. Anti-inflammatory agents include, but are not limited to, acetaminophen, diphenhydramine, meperidine, dexamethasone, pentasa, mesalazine, asacol, codeine phosphate, benorylate, fenbufen, naprosyn, diclofenac, etodolac and indomethacin, aspirin, and ibuprofen.

For treatment of cancers, chemotherapeutic agents can be used in combination with the antibodies, or antigen-binding fragments thereof, described herein. Chemotherapeutic agents include, but are not limited to, radioactive molecules, toxins (such as cytotoxins or cytotoxic agents) which include any agent that is detrimental to the viability of cells, agents, and liposomes or other vesicles containing chemotherapeutic compounds. Examples of suitable chemotherapeutic agents include, but are not limited to, 1-dehydrotestosterone, 5-fluorouracil decarbazine, 6-mercaptopurine, 6-thioguanine, actinomycin D, adriamycin, aldesleukin, an anti-α5β1 integrin antibody, alkylating agents, allopurinol sodium, altretamine, amifostine, anastrozole, anthramycin (AMC)), anti-mitotic agents, cisdichlorodiamine platinum (II) (DDP) cisplatin, diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytarabine, cytochalasin B, cytoxan, dacarbazine, dactinomycin, dactinomycin (formerly actinomycin), daunirubicin, daunorucbicin citrate, denileukin diftitox, dexrazoxane, dibromomannitol, dihydroxy anthracin dione, docetaxel, dolasetron mesylate, doxorubicin, dronabinol, E. coli L-asparaginase, eolociximab, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine, glucocorticoids, goserelin acetate, gramicidin D, granisetron, hydroxyurea, idarubicin, ifosfamide, interferon α-2b, irinotecan, letrozole, leucovorin calcium, leuprolide acetate, levamisole, lidocaine, lomustine, maytansinoid, mechlorethamine, medroxyprogesterone acetate, megestrol acetate, melphalan, mercaptipurine, mesna, methotrexate, methyltestosterone, mithramycin, mitomycin C, mitotane, mitoxantrone, nilutamide, octreotide acetate, ondansetron, paclitaxel, pamidronate disodium, pentostatin, pilocarpine, plimycin, polifeprosan 20 with carmustine implant, porfimer sodium, procaine, procarbazine, propranolol, rituximab, sargramostim, streptozotocin, tamoxifen, taxol, teniposide, tenoposide, testolactone, tetracaine, thioepa chlorambucil, thioguanine, thiotepa, topotecan, toremifene citrate, trastuzumab, tretinoin, valrubicin, vinblastine sulfate, vincristine sulfate, and vinorelbine tartrate, or any salt thereof. In some embodiments, the methods comprise administering at least one chemotherapeutic agent to the patient.

Any anti-angiogenic agent can be used in conjunction with the antibodies, or antigen-binding fragments thereof, described herein. In some embodiments, the anti-angiogenic agent is a VEGF antagonist or another VEGF receptor antagonist such as VEGF variants, soluble VEGF receptor fragments, aptamers capable of blocking VEGF or VEGFR, neutralizing anti-VEGFR antibodies, low molecule weight inhibitors of VEGFR tyrosine kinases and any combinations thereof. Alternately, or in addition, an anti-VEGF antibody may be co-administered to the patient.

The therapeutic regimen administered to the patient can vary depending on the patient's age, weight, and disease condition. The therapeutic regimen can continue for 2 weeks to indefinitely. In some embodiments, the therapeutic regimen is continued for about 2 weeks to about 6 months, from about 3 months to about 5 years, from about 6 months to about 1 or about 2 years, from about 8 months to about 18 months, or the like. The therapeutic regimen can be a non-variable dose regimen or a multiple-variable dose regimen.

The amount of the antibodies, or antigen-binding fragments thereof, described herein administered can depend upon a variety of factors including, but not limited to, the particular type of solid tumor treated, the stage of the solid tumor being treated, the mode of administration, the frequency of administration, the desired therapeutic benefit, and other parameters such as the age, weight and other characteristics of the patient. Determination of dosages effective to provide therapeutic benefit for specific modes and frequency of administration is within the capabilities of those skilled in the art. Dosages effective to provide therapeutic benefit may be estimated initially from in vivo animal models or clinical trials. Suitable animal models for a wide variety of diseases are known in the art. The antibodies, or antigen-binding fragments thereof, described herein may be administered by any route appropriate to the condition to be treated.

In some embodiments, the antibodies, or antigen-binding fragments thereof, described herein are provided as a lyophilized powder in a vial. The vials can contain about 100 mg, about 125 mg, about 150 mg, about 200 mg, about 250 mg, about 300 mg, or about 400 mg of the antibodies, or antigen-binding fragments thereof. Prior to administration, the lyophilized powder can be reconstituted with sterile water for injection (SWFI) or other suitable medium to provide a solution containing the antibodies, or antigen-binding fragments thereof, described herein. In some embodiments, the resulting reconstituted solution is further diluted with saline or other suitable medium for infusion and administered via, for example, an IV infusion twice every 7 days, once every 7 days, once every 14 days, once every 21 days, once every 28 days, once every 35 days, once every 42 days, once every 49 days, or once every 56 days. In some embodiments, for the first cycle, the infusion occurs over 90 minutes. In some embodiments, subsequent infusions are over 60 minutes.

In some embodiments, the antibodies, or antigen-binding fragments thereof, described herein are administered as an IV infusion once every 7 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the antibodies, or antigen-binding fragments thereof, described herein are administered as an IV infusion once every 14 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the antibodies, or antigen-binding fragments thereof, described herein are administered as an IV infusion once every 21 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg. In some embodiments, the antibodies, or antigen-binding fragments thereof, described herein are administered as an IV infusion once every 28 days at about 0.1 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 8.0 mg/kg, or about 10.0 mg/kg.

When administered adjunctive to or with other agents, such as other chemotherapeutic agents, the antibodies, or antigen-binding fragments thereof, described herein can be administered on the same schedule as the other agent(s), or on a different schedule. When administered on the same schedule, the antibodies, or antigen-binding fragments thereof, described herein can be administered before, after, or concurrently with the other agent. In some embodiments, where the antibodies, or antigen-binding fragments thereof, described herein are administered adjunctive to, or with, standards of care, the antibodies, or antigen-binding fragments thereof, can be initiated prior to commencement of the standard therapy, for example one day, several days, one week, several weeks, one month, or even several months before commencement of standard of care therapy. In some embodiments, where the antibodies, or antigen-binding fragments thereof, described herein are administered adjunctive to, or with, standards of care, the antibodies, or antigen-binding fragments thereof, described herein can be initiated after commencement of the standard therapy, for example one day, several days, one week, several weeks, one month, or even several months after commencement of standard of care therapy.

The dosing schedule for subcutaneous administration can vary from once every six months to daily depending on a number of clinical factors, including the type of disease, the severity of disease, and the patient's sensitivity to the antibodies, or antigen-binding fragments thereof, described herein.

The present disclosure also provides methods of treating Posterior Capsule Opacification (PCO), the methods comprising administering to a human patient in need thereof the antibodies, or antigen-binding fragments thereof, described herein. In some embodiments, the antibodies, or antigen-binding fragments thereof, are administered to the eye.

The present disclosure also provides methods of treating fibrosis, the methods comprising administering to a human patient in need thereof the antibodies, or antigen-binding fragments thereof, described herein. In some embodiments, the antibodies, or antigen-binding fragments thereof, are administered to an organ. In some embodiments, the organ is a kidney or lung.

In some embodiments, the antibodies, or antigen-binding fragments thereof, described herein can be used in methods of isolating and/or purifying cells, such as, for example, myofibroblast progenitors, by sorting cells that bind thereto. Such cells can rapidly migrate to wounds in the skin, lens, retina, and brain, thereby aiding in wound healing. Such cells have the potential to develop into contractile myofibroblasts. Isolated antigen-positive cells can be administered to a human or implanted into slowly healing or non-healing wounds, such as diabetic and decubitis ulcers and severe surgical resections, to facilitate wound closure.

In some embodiments, the antibodies, or antigen-binding fragments thereof, described herein can be used in methods of isolating neuroprotective cells by sorting cells that bind thereto. Cells that express antigen can increase in number in response to injury. Such cells can be administered to the retina and brain to reduce the death of neurons. Antigen-positive cells and/or the molecule(s) they produce can be injected or otherwise introduced to the subject following injury.

The oligonucleotides disclosed herein can comprise, for example, nucleotides or non-natural, or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophore-labeled nucleotides. The oligonucleotides disclosed herein can comprise DNA, RNA, or both DNA and RNA.

The oligonucleotides disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S—or N-alkynyl; or O-alkyl-O-alkyl, wherein the alkyl, alkenyl, and alkynyl may be substituted or unsubstituted C₁₋₁₀ alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to. C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to S′-2′. Various salts, mixed salts, and free acid forms are also included. Nucleotide substitutes also include peptide nucleic acids (PNAs).

Different lengths of oligonucleotides, different base compositions, different arrangement(s) of nucleotides, and/or different oligonucleotide densities on the antibody may have different effects allowing for the modulation of molecular properties and, ultimately, therapeutic efficacy or therapeutic index of biologics.

The chemistry of attachment of the oligonucleotide may also offer unique modulatory properties or flexibility to adaptation of the design of novel therapeutic entities whose properties are linked to both biodistribution as well as clearance rate.

The disclosed nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The nucleic acid molecules disclosed herein can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂),CH₃)|2, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the oligonucleotide has the nucleotide sequence set forth in SEQ ID NO:1.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning-A Laboratory Manual, 2nd ed., Cold Spring Harbor Press (1989), using commercially available reagents, except where otherwise noted. As used herein, numbering of immunoglobulin amino acid residues is carried out according to the immunoglobulin amino acid residue numbering system of Kabat et al., unless otherwise indicated.

To study the circulation and biodistribution of anti-ICAM/3DNA in vivo, anti-ICAM antibody was coupled to 3DNA outer branches. Since the outer branches of 3DNA are single-stranded DNA, a means of coupling anti-ICAM to 3DNA was to first conjugate anti-ICAM to an oligonucleotide whose sequence is complementary to that of 3DNA outer branches, so that antibody-oligo conjugate could be coupled to 3DNA simply through annealing by mixing them together. Since specific targeting of ICAM-1 had to be demonstrated, similar constructs using non-specific antibody (IgG control) were also comparatively studied. However, before coupling anti-ICAM-oligo or IgG-oligo to 3DNA for subsequent injection in mice of respective anti-ICAM/3DNA or anti-ICAM/3DNA, it was necessary to demonstrate that the conjugated species (anti-ICAM-oligo or IgG-oligo) were still able to behave as the original anti-ICAM and IgG, i.e. that they would target ICAM⁻¹, which is mostly expressed in the lungs, or serve as a non-specific control antibody, respectively. For this purpose, mice were injected with IgG, IgG-oligo, anti-ICAM, or anti-ICAM-oligo, and their circulation and biodistribution was then addressed and compared. Surprisingly, the results demonstrated that antibody-oligo species behaved differently from the original antibody species, where the presence of the oligo increased the targeting specificity of anti-ICAM.

The disclosed studies show that coupling of an antibody to a DNA oligonucleotide modifies the antibody behavior in an unexpected manner. For an example of a specific antibody, of which the monoclonal antibody that recognizes ICAM-1 is illustrative, conjugation to an oligonucleotide markedly increased both the absolute binding and binding specificity to the antibody's respective antigen (ICAM-1), which was demonstrated in vivo. In addition, antibody-oligo conjugates (both specific and non-specific) cleared from the circulation faster than the original non-conjugated antibodies and were distributed more profusely, which may serve to avoid systemic side effects of antibodies applied in vivo. This unexpected discovery holds significant translational potential to enhance the recognition/targeting ability of an antibody toward its antigen, which can be used as a means to enhance the research, therapeutic and/or diagnostic potential of said antibody, whether used alone or in combination with drugs, tracers, carriers, devices, materials, or cells for several purposes and applications, which may involve analytical in vitro systems, ex vivo tissues, cell cultures, or in vivo organisms.

### EXAMPLES

### Example 1: In Vivo Biodistribution of Non-Specific IgG Antibody, IgG Antibody-Oligonucleotide, Specific Anti-ICAM Antibody, and Anti-ICAM Antibody-Oligonucleotide

### Reagents:

Rat monoclonal immunoglobulin G Ab (IgG) against mouse ICAM-1 (anti-ICAM) was clone YNI, produced in a respective hybridoma from the American Type Culture Collection (Manassas, VA). Non-specific rat IgG Ab (called IgG hereafter) was obtained from Jackson Immunoresearch (Pike West Grove, PA). DNA oligonucleotide (72-mer) modified with thiol at the 5′ terminus was obtained from Oligo Factory (Holliston, MA). Pierce Bond-Breaker TCEP Solution, Pierce LC-SMCC Crosslinker, Pierce Zeba Spin Columns (7 k molecular weight cut-off, MWCO), Pierce Thiophilic Adsorption Resin, Amicon spin filters (10 k MWCO), Pierce BCA Protein Assay Kit and Heterobifunctional Pierce Crosslinking Kit, bovine serum albumin (BSA), and TCA were obtained from Fisher Scientific (Kerrville, TX). Iodogen iodination tubes were obtained from Pierce (Rockford, IL) and BioSpin Tris Columns were obtained from BioRad (Hercules, CA). Na¹²⁵I was obtained from Perkin-Elmer (Waltham, MA). All other reagents were obtained from Sigma Chemical (St. Louis, MO).

### Antibody-Oligonucleotide Conjugates:

Antibody-oligonucleotide (Ab-oligo) conjugations for anti-ICAM or non-specific control IgG Abs were performed using N-hydroxysuccinimide (NHS)-maleimide chemistry In brief, 72-mer DNA oligonucleotide with a 5′-thiol modification was first reduced by adding 50 mM tris (2-carboxyethyl)phosphine (TCEP) and incubating at 25° C. for 1 hour. Excess TCEP was removed by ethanol precipitation and the oligonucleotide resuspended in phosphate buffer saline (PBS)+5 mM ethylenediaminetetraacetic acid (EDTA), pH 7.2. In parallel, Ab was reacted with LC-SMCC crosslinker (succinimidyl 4-(N-maleimidomethyl) cyclohexane-1-carboxy-(6-amidocaproate) in excess at 25° C. for 1 hour. Zeba spin columns, equilibrated in PBS+5 mM EDTA pH 7.2, were used to remove unreacted LC-SMCC cross-linker from the Ab-LC-SMCC reaction as per the vendor's instructions. Reduced oligonucleotide was then added to the Ab-LC-SMCC and the conjugation reaction was allowed to incubate at 25° C. for 12 hours. Thiophilic adsorption chromatography was utilized to remove excess unreacted thiol oligonucleotide from the Ab-oligonucleotide conjugate and fractions containing the conjugate were pooled and concentrated using Amicon 10 kDa MWCO spin filters. The final protein concentration of the Ab-oligonucleotide conjugate was determined using a (bicinchoninic acid) BCA Protein Assay kit with bovine gamma globulin standards.

### Radiolabeling of Antibodies and Antibody-Oligo Conjugates:

Abs (non-oligonucleotide) or Ab-oligonucleotide conjugates were 1251-radiolabeled using Na¹²⁵I and iodogen-coated iodination tubes. 20 μCi of Na¹²⁵I was incubated at 4° C. for 5 minutes in iodination tubes containing 100 μL of 1 μg/μL Ab in PBS. Samples were then subjected to size exclusion chromatography in 6 kDa cutoff Tris columns and centrifuged at 1000×g for 4 minutes to eliminate non-reacted free ¹²⁵I. The final concentrations of the resulting ¹²⁵I-Abs or ¹²⁵I-Ab-oligonucleotide conjugates were measured using Bradford protein assay and BSA standard curve. The radioactivity (counts of detected radioactive events per minute, CPM) of the samples, expressed as CPM per μg protein, and the presence of free ¹²⁵I remnants were then measured in a γ-counter (2470 Wizard²™, Perkin Elmer, Waltham, MA). For this, 2 μL of ¹²⁵I-Ab or ¹²⁵I-Ab-oligonucleotide were diluted in 3% BSA in PBS, resulting in 1 mL total volume and then measured in a γ-counter. Next, 200 μL of TCA was added (final TCA concentration=17%, v/v), the sample was vortexed and incubated for 15 minutes at room temperature. Then, the sample was centrifuged at 2418×g for 5 minutes to precipitate ¹²⁵I-Ab-oligonucleotide and separate free 1251 in the supernatant. A 600 μL aliquot (half the total reaction volume) of the supernatant was then measured in a γ-counter, to estimate the presence of free ¹²⁵I in the samples to be injected in mice using the following equation: Free Iodine CPM=Total CPM-(2 ×supernatant CPM).

Mouse model:

Eight-week old C57BL/6 wild type male mice were obtained from Jackson Laboratory (Bar Harbor, ME). The mice were provided food and water ad libitum and used in experiments as received and at about 25 g body weight (BW). Mice were intraperitoneally anesthetized with a mixture of 100 mg/kg of BW ketamine and 10 mg/kg of BW xylazine for each in vivo experiment. All animal experiments were conducted in compliance with regulations and under institutionally approved protocols.

### Circulation and Biodistribution:

Anesthetized C57BL/6 mice were injected with ¹²³I-labeled antibodies or antibody-oligonucleotide conjugates. Blood samples were collected at the indicated time points and at sacrifice 60 minutes after injection, as indicated, followed by collection and weighing of main organs (e.g., brain, heart, kidneys, liver, lungs, and spleen), organ homogenization, and sample precipitation with trichloroacetic acid (TCA) to eliminate any potential free ¹²⁵I in the samples. Radioactivity measurements obtained using a γ-counter were then utilized to calculate the % injected dose (% ID), where the injected dose is the dose measured prior to injection minus the dose remnant in the syringe after the injection. The % injected dose per gram of organ (% ID/g), which compares relative accumulation in organs with different weights and, thus, reflects organ concentration, was also calculated. The localization ratio (LR= % ID/g in an organ: % ID/g in blood) to express the organ-to-blood distribution; and the specificity index (SI), calculated as the % ID/g of a species divided by the % ID/g of the other species, to compare them.

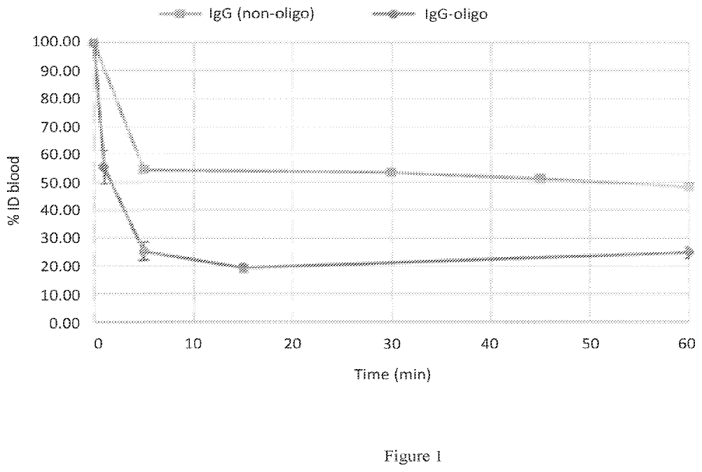

FIG. 1 shows IgG-oligonucleotide enhances removal from the bloodstream. Mice were injected intravenously with non-specific IgG control antibody or the same antibody conjugated to an oligonucleotide (IgG-oligo). The entities were also radiolabeled with iodine¹²⁵ to trace their circulation up to 60 minutes after administration. Data were calculated as mean±standard error of the mean. The parameter shown is the % of the injected dose (% ID), reflective of absolute biodistribution values in circulation. FIG. 1 demonstrates faster clearance from the circulation for non-specific IgG-oligo compared to IgG, which may help reduce possible systemic side effects associated to the use of antibodies.

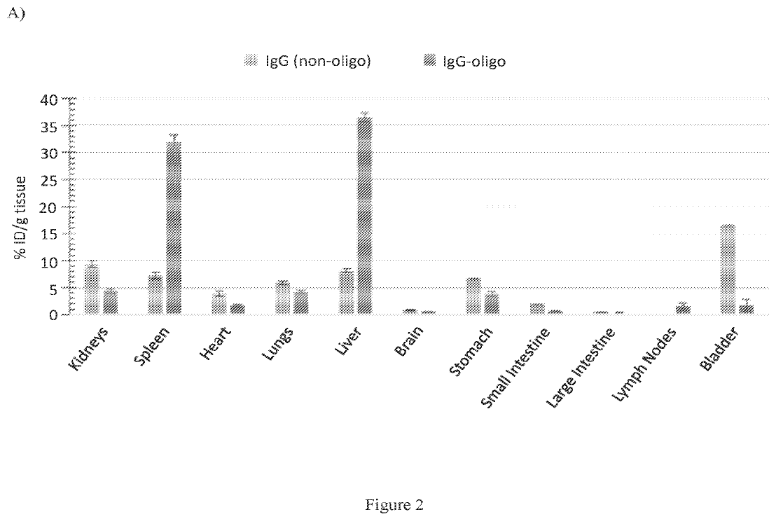

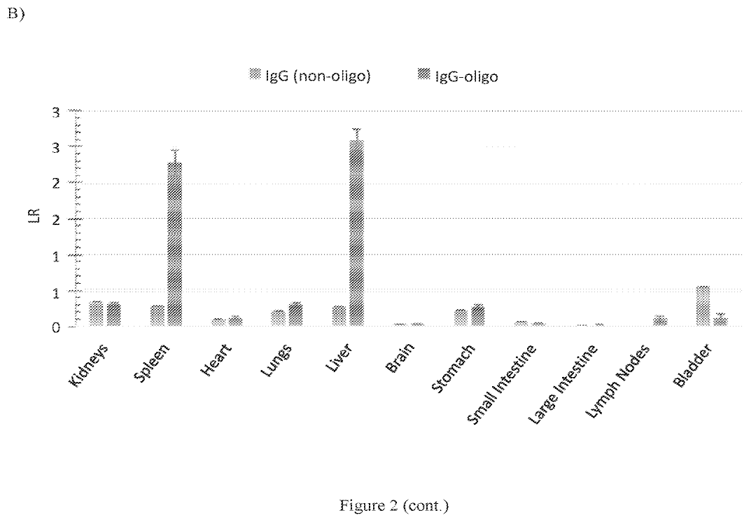

FIG. 2 shows IgG-oligonucleotide enhances spleen and liver clearance. Mice were injected intravenously with non-specific IgG control antibody or the same antibody conjugated to an oligonucleotide (IgG-oligo). The entities were radiolabeled with iodine¹²⁵ to trace their body distribution 60 minutes after administration. Data were calculated as mean±standard error of the mean. The parameters shown are the % of the injected dose per gram of organ (% ID/g), reflective of concentration in organs (Panel A), and the localization ratio (LR), which is the % ID/g in an organ divided by the % ID/g in blood (Panel B), reflective of the tissue-to-blood ratio. FIG. 2 demonstrates increased liver and spleen accumulation of non-specific IgG-oligo compared to IgG, which can help clear the antibody-oligo from the body through these clearance organs and attenuate possible side effects in other organs. Also, there is some decrease in the level of IgG-oligo in the bladder, kidney, and stomach. This may be due to enhanced clearance in the liver and spleen or faster excretion through the renal and gastric routes, so that lesser amounts are found at this time in these organs, which is adequate to remove the antibody from the body and avoid side effects.

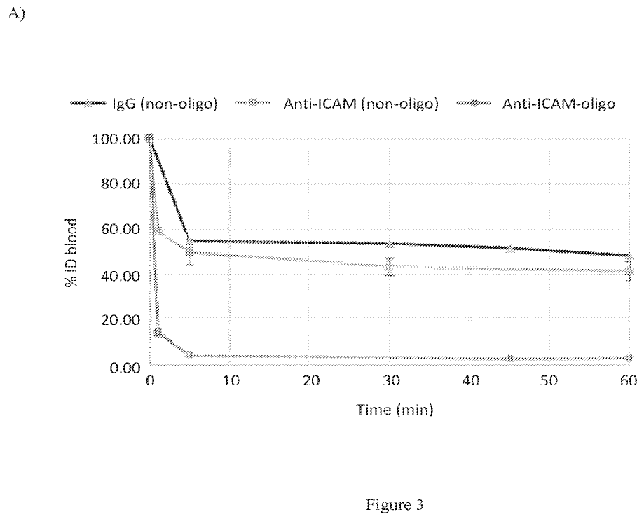

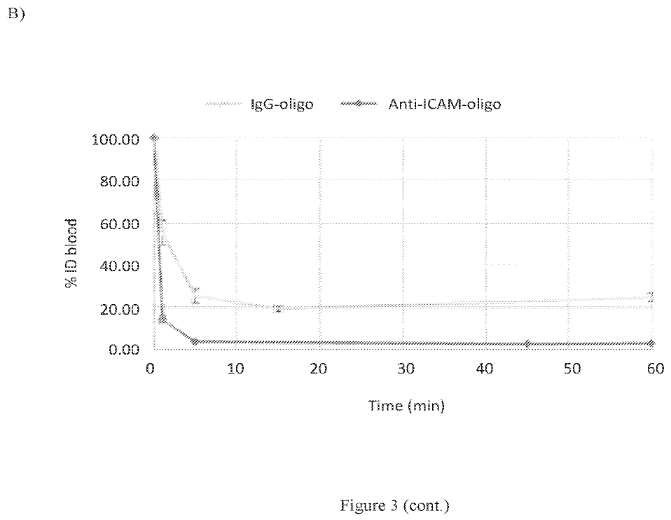

FIG. 3 shows anti-ICAM-oligonucleotide is rapidly removed from the bloodstream. Mice were injected intravenously with non-specific IgG control antibody, ICAM-1 specific anti-ICAM antibody, or anti-ICAM-oligo conjugate (Panel A), or anti-ICAM-oligo or IgG-oligo conjugates (Panel B). In both cases, the entities were radiolabeled with iodine¹²⁵ to trace their circulation up to 60 minutes after administration. Data were calculated as mean±standard error of the mean. The parameter shown is the % of the injected dose (% ID), reflective of absolute biodistribution values in circulation. FIG. 3 demonstrates specific anti-ICAM antibody faster clearance from the circulation when conjugated to oligonucleotide compared to non-oligonucleotide anti-ICAM, which should also serve to protect from systemic side effects, as described above. In addition, faster clearance of anti-ICAM-oligonucleotide compared to IgG-oligonucleotide indicates that this protective effect is more apparent for an antibody that recognizes a specific antigen. This is relevant as specific antibodies are the ones recognizing antigens, therefore, the ones intended to be used in research, diagnostic, or therapeutic settings.

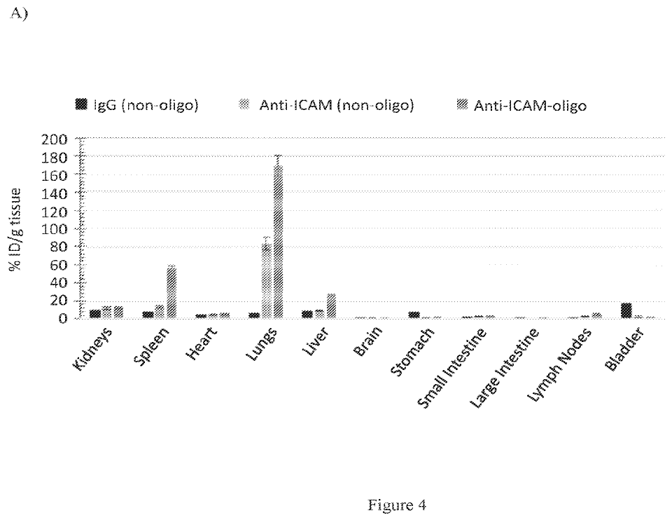

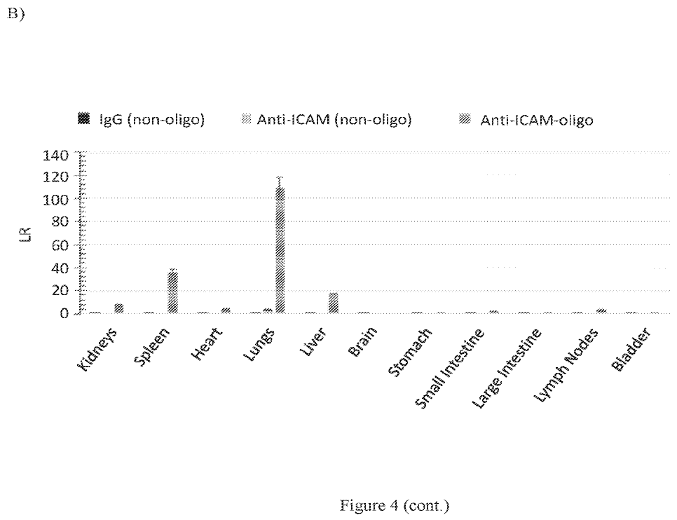

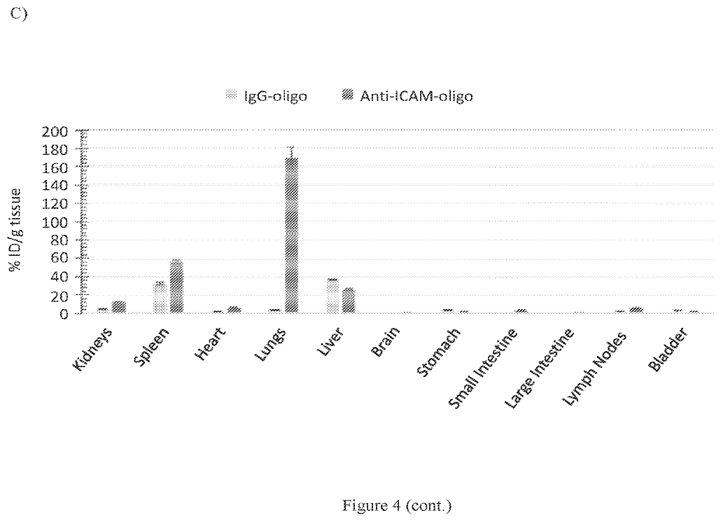

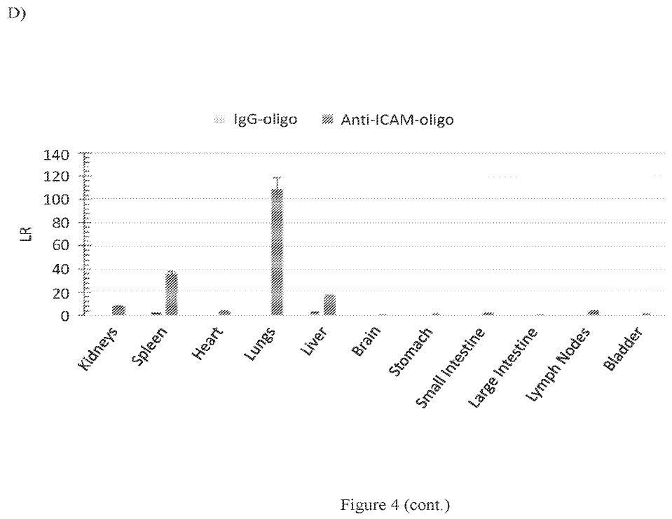

FIG. 4 shows anti-ICAM-oligo enhances lung targeting. Mice were injected intravenously with non-specific IgG control antibody, ICAM-1 specific anti-ICAM antibody, or anti-ICAM-oligo conjugate (Panel A and Panel B) or anti-ICAM-oligo or IgG-oligo conjugates (Panel C and Panel D). In all cases, the entities were radiolabeled with iodine¹²⁵ to trace their body distribution 60 minutes after administration. Data were calculated as mean±standard error of the mean. The parameters shown are the percent of the injected dose per gram of organ (% ID/g), reflective of concentration in organs (Panel A and Panel C), and the localization ratio (LR), which is the % ID/g in an organ divided by the % ID/g in blood, reflective of the tissue-to-blood ratio (Panel B and Panel D). FIG. 4 demonstrates that specific anti-ICAM antibody targets the lungs (main site for ICAM-1 expression) markedly better compared to anti-ICAM control and much greater than non-specific IgG (conjugated or not to the oligonucleotide), demonstrating enhanced absolute targeting and specificity.

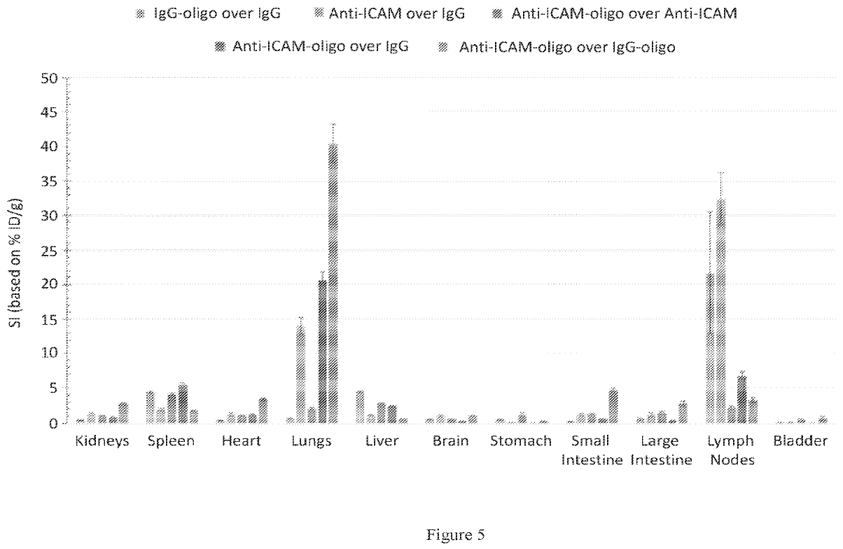

FIG. 5 shows lung targeting of anti-ICAM-oligonucleotide is highly specific for the lung compared to other formulations and provided minimal accumulation in lymph nodes. Mice were injected intravenously with non-specific IgG control antibody, ICAM-1 specific anti-ICAM antibody, or respective antibodies conjugated to an oligonucleotide (oligo), where these entities were radiolabeled with iodine¹²⁵ to trace their body distribution 60 minutes after administration. Data were calculated as mean±standard error of the mean. The parameters shown is the specificity index (SI) compares the biodistribution of the two species indicated in the graph key, calculated as the % ID/g of a species divided by the % ID/g of the other species, reflective of specificity. FIG. 5 demonstrates enhanced specificity toward the main site for ICAM-1 expression (the lungs) offered by anti-ICAM-oligo as compared to all other species injected.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1-130. (canceled)

131. A method of treating a disease in a subject, the method comprising administering to the subject in need thereof a physiologically acceptable amount of an enhanced targeting composition, wherein the enhanced targeting composition comprises an antibody, or antigen-binding fragment thereof, conjugated to an oligonucleotide,
wherein the antibody, or antigen-binding fragment thereof, conjugated to the oligonucleotide binds an antigen to a greater extent or faster compared to an identical antibody, or antigen-binding fragment thereof, that is not conjugated to an identical oligonucleotide;
wherein the antibody, or antigen-binding fragment thereof, conjugated to an oligonucleotide is cleared from blood of the subject faster after administration to the subject compared to an identical antibody, or antigen-binding fragment thereof, not conjugated to an identical oligonucleotide; and
wherein the antibody, or antigen-binding fragment thereof, conjugated to the oligonucleotide is not hybridized to a DNA dendrimer.

132. The method of claim 131, wherein the administration to the subject is intravenous administration.

133. The method of claim 131, wherein the antibody, or antigen-binding fragment thereof, conjugated to an oligonucleotide has decreased non-specific binding compared to an identical antibody, or antigen-binding fragment thereof, not conjugated to an identical oligonucleotide.

134. The method of claim 131, wherein the administration to the subject results in a decrease in side effects compared to an identical antibody, or antigen-binding fragment thereof, not conjugated to an identical oligonucleotide.

135. The method of claim 131, wherein the enhanced targeting composition further comprises an effector moiety covalently linked to the antibody, or antigen-binding fragment thereof,
wherein the effector moiety is selected from a detectable label, a cytotoxic agent, a chemotherapeutic agent, a nucleic acid molecule, an antineoplastic agent, a drug, a toxin, a biologically active protein, another antibody or antigen-binding fragment thereof, a synthetic or naturally occurring polymer, a radionuclide, a radioisotope, a chelated metal, a nanoparticle, a reporter group, a drug moiety, a polypeptide, a signaling molecule, a thrombotic agent, an anti-angiogenic agent, a biological response modifier, or a chemotherapeutic agent; and
wherein the covalent linkage does not affect the binding of the antibody, or antigen-binding fragment thereof, to an antigen.

136. The method of claim 135, wherein the effector moiety increases a half-life of the composition in vivo or enhances delivery of the antibody or antigen-binding fragment across an epithelial barrier.

137. The method of claim 131, wherein the enhanced targeting composition further comprises a poly(ethyleneglycol) (PEG) moiety linked to the antibody, or antigen-binding fragment thereof.

138. The method of claim 131, wherein the antibody, or antigen-binding fragment thereof, comprises a polyclonal antibody or antigen-binding fragment thereof, a monoclonal antibody or antigen-binding fragment thereof, or a genetically engineered or otherwise modified antibody or antigen-binding fragment thereof.

139. The method of claim 131, wherein the antibody, or antigen-binding fragment thereof, comprises a chimeric antibody, a humanized antibody, a primatized antibody, or a heteroconjugate antibody.

140. The method of claim 131, wherein the antibody, or antigen-binding fragment thereof, comprises a Fab, a Fab′, a F(ab′)₂, a Fv, a scFv, a scFv-Fc, a diabody, a bispecific diabody, a trispecific triabody, a minibody, a monospecific Fab₂, a bispecific Fab₂, a trispecific Fab₃, a nanobody, a IgNAR, a V-NAR, an hcIgG, or a VhH fragment.

141. The method of claim 131, wherein the antibody, or antigen-binding fragment thereof, is a therapeutic antibody, wherein the therapeutic antibody comprises 3F8, 8H9, abagovomab, abciximab, abituzumab, abrezekimab, abrilumab, actoxumab, adalimumab, adecatumumab, aducanumab, afascvikumab, afclimomab, alacizumab pegol, alemtuzumab, alirocumab, altumomab pentetate, amatuximab, amivantamab, anatumomab mafenatox, andecaliximab, anctumab ravtansine, anifrolumab, ansuvimab, anrukinzumab, apolizumab, aprutumab ixadotin, arcitumomab, ascrinvacumab, asclizumab, atezolizumab, atidortoxumab, atinumab, atoltivimab, maftivimab, odesivimab, atorolimumab, avelumab, azintuxizumab vedotin, bamlanivimab, bapincuzumab, basiliximab, bavituximab, BCD-100, bectumomab, begelomab, belantamab mafodotin, belimumab, bemarituzumab, benralizumab, berlimatoxumab, bermekimab, bersanlimab, bertilimumab, besilesomab, bevacizumab, bezlotoxumab, biciromab, bimagrumab, bimckizumab, birtamimab, bivatuzumab, blesclumab, blinatumomab, blontuvetmab, blosozumab, bococizumab, brazikumab, brentuximab vedotin, briakinumab, brodalumab, brolucizumab, brontictuzumab, burosumab, cabiralizumab, camidanlumab tesirine, camrelizumab, canakinumab, cantuzumab mertansine, cantuzumab ravtansine, caplacizumab, casirivimab, capromab, carlumab, carotuximab, catumaxomab, a cBR96-doxorubicin immunoconjugate, cedelizumab, cemiplimab, cergutuzumab amunaleukin, certolizumab pegol, cetrelimab, cetuximab, cibisatamab, cirmtuzumab, citatuzumab bogatox, cixutumumab, clazakizumab, clenoliximab, clivatuzumab tetraxetan, codrituzumab, cofctuzumab pelidotin, coltuximab ravtansine, conatumumab, concizumab, cosfroviximab, crenczumab, crizanlizumab, crotedumab, CR6261, cusatuzumab, dacetuzumab, daclizumab, dalotuzumab, dapirolizumab pegol, daratumumab, dectrekumab, demcizumab, denintuzumab mafodotin, denosumab, depatuxizumab mafodotin, derlotuximab biotin, detumomab, dezamizumab, dinutuximab, dinutuximab beta, diridavumab, domagrozumab, dorlimomab aritox, dostarlimab, drozitumab, DS—8201, duligotuzumab, dupilumab, durvalumab, dusigitumab, duvortuxizumab, ecromeximab, cculizumab, cdobacomab, edrecolomab, cfalizumab, cfungumab, eldelumab, elezanumab, elgemtumab, clotuzumab, elsilimomab, emactuzumab, emapalumab, emibetuzumab, emicizumab, enapotamab vedotin, enavatuzumab, enfortumab vedotin, enlimomab pegol, enoblituzumab, enokizumab, enoticumab, ensituximab, epcoritamab, epitumomab cituxetan, cpratuzumab, eptinezumab, erenumab, erlizumab, ertumaxomab, ctaracizumab, etesevimab, ctigilimab, ctrolizumab, evinacumab, cvolocumab, exbivirumab, fanolesomab, faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab, foralumab, foravirumab, fremanczumab, fresolimumab, frovocimab, frunevetmab, fulranumab, futuximab, galcanezumab, galiximab, gancotamab, ganitumab, gantenerumab, gatipotuzumab, gavilimomab, gedivumab, gemtuzumab ozogamicin, gevokizumab, gilvetmab, gimsilumab, girentuximab, glembatumumab vedotin, golimumab, gomiliximab, gosuranemab, guselkumab, ianalumab, ibalizumab, IBI308, ibritumomab tiuxetan, icrucumab, idarucizumab, ifabotuzumab, igovomab, iladatuzumab vedotin, IMAB362, imalumab, imaprelimab, imciromab, imdevimab, imgatuzumab, inclacumab, indatuximab ravtansine, indusatumab vedotin, inebilizumab, infliximab, intetumumab, inolimomab, inotuzumab ozogamicin, ipilimumab, Iomab-B, iratumumab, isatuximab, iscalimab, istiratumab, itolizumab, ixekizumab, keliximab, labetuzumab, lacnotuzumab, ladiratuzumab vedotin, lampalizumab, lanadelumab, landogrozumab, laprituximab emtansine, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab vedotin, ligelizumab, loncastuximab tesirine, losatuxizumab vedotin, lilotomab satetraxetan, lintuzumab, lirilumab, lodelcizumab, lokivetmab, lorvotuzumab mertansine, lucatumumab, lulizumab pegol, lumiliximab, lumretuzumab, lupartumab, lupartumab amadotin, lutikizumab, maftivimab, mapatumumab, margetuximab, marstacimab, maslimomab, mavrilimumab, matuzumab, mepolizumab, metelimumab, milatuzumab, minretumomab, mirikizumab, mirvetuximab soravtansine, mitumomab, modotuximab, mogamulizumab, monalizumab, morolimumab, mosunetuzumab, motavizumab, moxctumomab pasudotox, muromonab-CD3, nacolomab tafenatox, namilumab, naptumomab estafenatox, naratuximab emtansine, narnatumab, natalizumab, navicixizumab, navivumab, naxitamab, nebacumab, necitumumab, nemolizumab, NEOD001, nerelimomab, nesvacumab, netakimab, nimotuzumab, nirsevimab, nivolumab, nofetumomab merpentan, obiltoxaximab, obinutuzumab, ocaratuzumab, ocrelizumab, odesivimab, odulimomab, ofatumumab, olaratumab, oleclumab, olendalizumab, olokizumab, omalizumab, omburtamab, OMS721, onartuzumab, ontuxizumab, onvatilimab, opicinumab, oportuzumab monatox, oregovomab, orticumab, otelixizumab, otilimab, otlertuzumab, oxelumab, ozanezumab, ozoralizumab, pagibaximab, palivizumab, pamrevlumab, panitumumab, pankomab, panobacumab, parsatuzumab, pascolizumab, pasotuxizumab, pateclizumab, patritumab, PDR001, pembrolizumab, pemtumomab, perakizumab, pertuzumab, pexelizumab, pidilizumab, pinatuzumab vedotin, pintumomab, placulumab, prezalumab, plozalizumab, pogalizumab, polatuzumab vedotin, ponczumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab, raxibacumab, ravagalimab, ravulizumab, refanezumab, regavirumab, regdanvimab, relatlimab, remtolumab, reslizumab, rilotumumab, rinucumab, risankizumab, rituximab, rivabazumab pegol, robatumumab, SII RMAb, roledumab, romilkimab, romosozumab, rontalizumab, rosmantuzumab, rovalpituzumab tesirine, rovelizumab, rozanolixizumab, ruplizumab, SA237, sacituzumab govitecan, samalizumab, samrotamab vedotin, sarilumab, satralizumab, satumomab pendetide, secukinumab, selicrelumab, seribantumab, setoxaximab, setrusumab, sevirumab, sibrotuzumab, SGN-CD19A, SHP647, sifalimumab, siltuximab, simtuzumab, siplizumab, sirtratumab vedotin, sirukumab, sofituzumab vedotin, solanezumab, solitomab, sonepcizumab, sontuzumab, spartalizumab, stamulumab, sulesomab, suptavumab, sutimlimab, suvizumab, suvratoxumab, tabalumab, tacatuzumab tetraxctan, tadocizumab, tafasitamab, talacotuzumab, talizumab, talquetamab, tamtuvetmab, tanczumab, taplitumomab paptox, tarextumab, tavolimab, teclistamab, tefibazumab, telimomab aritox, telisotuzumab, telisotuzumab vedotin, tenatumomab, teneliximab, teplizumab, tepoditamab, teprotumumab, tesidolumab, tetulomab, tezepelumab, TGN1412, tibulizumab, tildrakizumab, tigatuzumab, timigutuzumab, timolumab, tiragolumab, tiragotumab, tislelizumab, tisotumab vedotin, TNX-650, tocilizumab, tomuzotuximab, toralizumab, tosatoxumab, tositumomab, tovetumab, tralokinumab, trastuzumab, trastuzumab duocarmazine, trastuzumab emtansine, TRBS07, tregalizumab, tremelimumab, trevogrumab, tucotuzumab celmoleukin, tuvirumab, ublituximab, ulocuplumab, urelumab, urtoxazumab, ustekinumab, utomilumab, vadastuximab talirine, vanalimab, vandortuzumab vedotin, vantictumab, vanucizumab, vapaliximab, varisacumab, varlilumab, vatelizumab, vedolizumab, veltuzumab, vepalimomab, vesencumab, visilizumab, vobarilizumab, volociximab, vonlerolizumab, vopratelimab, vorsetuzumab mafodotin, votumumab, vunakizumab, xentuzumab, XMAB-5574, zalutumumab, zanolimumab, zatuximab, zenocutuzumab, ziralimumab, zolbetuximab, or zolimomab aritox.

142. The method of claim 131, wherein the oligonucleotide comprises a non-natural nucleotide, a modified nucleotide, a nucleotide analog, or a nucleotide substitute.

143. The method of claim 142, wherein the non-natural nucleotide, the modified nucleotide, the nucleotide analog, or the nucleotide substitute comprises a modified base, a modified sugar, or a modified phosphate group.

144. The method of claim 142, wherein the non-natural nucleotide comprises a dideoxynucleotide, a biotinylated nucleotide, an aminated nucleotide, a deaminated nucleotide, an alkylated nucleotide, a benzylated nucleotide, or a fluorophore-labeled nucleotide.

145. The method of claim 143, wherein the modified sugar comprises a sugar mimetic.

146. The method of claim 143, wherein the modified phosphate group comprises a modification such that a linkage between a first nucleotide and a second nucleotide comprises a phosphorothioate, a chiral phosphorothioate, a phosphorodithioate, a phosphotriester, an aminoalkylphosphotriester, a methyl phosphonate, an alkyl phosphonate, a phosphinate, a phosphoramidate, a thionophosphoramidates, a thionoalkylphosphonates, a thionoalkylphosphotriester, or a boranophosphate.

147. The method of claim 142, wherein the nucleotide substitute comprises a peptide nucleic acid (PNA).

148. The method of claim 147, wherein the greater extent comprises an increase in affinity or avidity.

149. The method of claim 131, wherein the oligonucleotide has a sequence of SEQ ID NO: 1.

150. The method of claim 131, wherein the antibody, or antigen binding fragment thereof, in an anti-ICAM antibody, or antigen binding fragment thereof.


SEQUENCE LISTING (1 sequence)

SEQ ID NO: 1 - DNA, 72 bp, Artificial Sequence
  Oligo sequence
ttttttccaccttagagtacaaacggaacacgagaatttttaccctacagagtaacctagattgatcaaaca